(19) United States
(12) Patent Application Publication
(10) Pub. No.: US 2025/0043081 A1
(43) Pub. Date: Feb. 6, 2025

(54) OLIGONUCLEOTIDE DENDRIMERS FOR DYNAMIC AND FUNCTIONAL COLLOIDAL CRYSTAL ENGINEERING

(71) Applicant: NORTHWESTERN UNIVERSITY, Evanston, IL (US)

(72) Inventors: Chad A. Mirkin, Wilmette, IL (US); Max Everett Distler, Evanston, IL (US); Ho Fung Cheng, Evanston, IL (US); Kyle Joseph Gibson, Evanston, IL (US)

(21) Appl. No.: 18/717,280

(22) PCT Filed: Dec. 5, 2022
(86) PCT No.: PCT/US22/51847

§ 371 Date: Jun. 6, 2024

Related U.S. Application Data
(60) Provisional application No. 63/286,524, filed on Dec. 6, 2021.

(51) Int. Cl. C08G 83/00; C12N 15/11

(52) U.S. Cl. CPC C08G 83/004; C08G 83/008; C12N 15/11; C12N 2310/3519; C12N 2310/52

(57) ABSTRACT

The present disclosure is directed to multicomponent assemblies (e.g., crystalline structures) using oligonucleotide dendrimers and spherical nucleic acids (SNAs). The disclosure also provides methods of forming the multicomponent assemblies.

### CROSS-REFERENCE TO RELATED APPLICATIONS

This application claims the priority benefit under 35 U.S.C. § 119 (e) of U.S. Provisional Patent Application No. 63/286,524, filed Dec. 6, 2021, which is incorporated herein by reference in its entirety.

### STATEMENT OF GOVERNMENT INTEREST

This invention was made with government support under grant numbers CHE1709888-001 awarded by National Science Foundation, FA8650-15-2-5518 awarded by Air Force Research Laboratory (AFRL), N00014-16-1-3117 awarded by Department of Defense, Department of the Navy, Office of Naval Research, and FA9550-17-1-0348 awarded by Department of Defense, Department of the Air Force, Air Force Office of Scientific Research. The government has certain rights in the invention.

### SUMMARY

Nanoparticle assembly is the foundation for many modern materials and next generation technologies. Nevertheless, control over such assemblies is greatly limited with most techniques. The present disclosure provides unprecedented programmability at the nanoscale, paving the way for the development of new, functional, and responsive materials.

Assembling nanoparticles into programmable and functional colloidal crystals is plagued by inherent dispersity across nanoscale building blocks, a lack of specificity in the interactions they can form with one another, the difficulty in restructuring building block arrangement post-synthesis, and the limited ways in which multiple components can be combined into a single assembly. Oligonucleotide chemistry addresses the issue of specificity, enabling the programmable assembly of nanomaterials facilitated by hydrogen bonding between complementary strands. Nevertheless, functionalizing nanoparticle surfaces with oligonucleotide (e.g., DNA) ligands yields disperse building blocks without valency or position control and with insufficient options for multicomponent assembly. Prior to the present disclosure, there were limited modular approaches to synthesizing structurally defined and reconfigurable nanoscale building blocks with a wide range of sizes and valences, greatly limiting the ability to design precisely crafted and switchable materials. The technology described herein addresses these needs by providing a novel approach to structurally defined oligonucleotide (e.g., DNA) dendrimers that can be reversibly (dis) assembled via simple strand displacement reactions. These oligonucleotide (e.g., DNA) dendrimers are capable of templating extended assemblies of nanoparticle with unprecedented control over the resulting material s crystal structure, lattice parameter, colloidal bonding character, and thermostability. Furthermore, this approach enables the development of multicomponent and multifunctional materials that can have a significant impact on modern technology by incorporating several different types of nanoparticles into a defined and programmable colloidal crystal.

Accordingly, the present disclosure is generally directed to the synthesis of molecularly defined, oligonucleotide (e.g., DNA)-based, nanoscale building blocks that can be used to program crystal engineering through oligonucleotide (e.g., DNA) interactions. These oligonucleotide dendrimers provide a novel route to control building block valency, anisotropy, orthogonality and size to form extended and responsive nanoparticle assemblies with programmable phase symmetry, lattice parameter, and crystal habit, enabling the development of new functional materials from nanoscale building blocks. Furthermore, this approach enables the formation of multicomponent assemblies and multifunctional materials.

Applications of the technology described herein include, but are not limited to:
Crystal engineering over a vast array of lattice parameters, phase symmetry, crystal habit, and thermostability;
The formation of responsive and reconfigurable materials capable of reorganizing as a result of environmental stimuli or the addition pre-programmed oligonucleotide (e.g., DNA) strand;
The design of multicomponent nanoparticle assemblies that contain new catalytic, magnetic, plasmonic, optical, and electronic properties as a result of their controlled assembly into one material;
The synthesis of discrete nanoparticle assemblies with novel organizations and distinct geometries

Advantages of the technology provided herein include, but are not limited to:
Enables fine-tuned and modular control over the specific architecture of each building block.
Allows for precise tuning of building block valency, size, and oligonucleotide (e.g., DNA) distribution, which in turn can be used to program specific assembly features.
Provide an unprecedented level of reconfigurability of nanoscale architecture, enabling colloidal crystal structure, colloidal bonding, as well as material's thermostability to be switched at the same time via simple addition of chemical stimuli.
While multicomponent assemblies are difficult to synthesize as a result of a lack of programmable techniques, this method enables such control independent of nanoparticle size, shape, and composition.

Accordingly, in some aspects the disclosure provides an oligonucleotide dendrimer comprising: a template core comprising a first oligonucleotide and a second oligonucleotide that are hybridized to each other such that the template core comprises a first overhang and a second overhang, wherein the first overhang and the second overhang are arranged in a target orientation; a first oligonucleotide dendron comprising an oligonucleotide stem linked to a plurality of oligonucleotide branches, wherein the oligonucleotide stem is hybridized to the first overhang; and a second oligonucleotide dendron comprising an oligonucleotide stem linked to a plurality of oligonucleotide branches, wherein the oligonucleotide stem is hybridized to the second overhang. In some embodiments, the plurality of oligonucleotide branches of the first oligonucleotide dendron and the plurality of oligonucleotide branches of the second oligonucleotide dendron have identical nucleotide sequences. In some embodiments, the plurality of oligonucleotide branches of the first oligonucleotide dendron and the plurality of oligonucleotide branches of the second oligonucleotide dendron have different nucleotide sequences. In some embodiments, in the target orientation the first overhang and the second overhang are oppositely disposed. In further embodiments, the template core comprises a third oligonucleotide that is hybridized to the first oligonucleotide and the second oligonucleotide such that the template core comprises a third overhang, wherein the first overhang, the second overhang, and the third overhang are arranged in a target orientation, the oligonucleotide dendrimer further comprising a third oligonucleotide dendron comprising an oligonucleotide stem linked to a plurality of oligonucleotide branches, wherein the oligonucleotide stem is hybridized to the third overhang. In some embodiments, the plurality of oligonucleotide branches of the first oligonucleotide dendron, the plurality of oligonucleotide branches of the second oligonucleotide dendron, and the plurality of oligonucleotide branches of the third oligonucleotide dendron have identical nucleotide sequences. In further embodiments, the plurality of oligonucleotide branches of the first oligonucleotide dendron, the plurality of oligonucleotide branches of the second oligonucleotide dendron, and the plurality of oligonucleotide branches of the third oligonucleotide dendron have different nucleotide sequences. In some embodiments, the plurality of oligonucleotide branches of the first oligonucleotide dendron and the plurality of oligonucleotide branches of the second oligonucleotide dendron have identical nucleotide sequences, but the plurality of oligonucleotide branches of the third oligonucleotide dendron has nucleotide sequences that are different than the plurality of oligonucleotide branches of the first oligonucleotide dendron and the plurality of oligonucleotide branches of the second oligonucleotide dendron. In some embodiments, in the target orientation the first overhang, the second overhang, and the third overhang are in a radial orientation. In some embodiments, the template core comprises a fourth oligonucleotide and hybridization of the first oligonucleotide, the second oligonucleotide, the third oligonucleotide, and the fourth oligonucleotide creates a fourth overhang, wherein the first overhang, the second overhang, the third overhang, and the fourth overhang are arranged in a target orientation, the oligonucleotide dendrimer further comprising a fourth oligonucleotide dendron comprising an oligonucleotide stem linked to a plurality of oligonucleotide branches, wherein the oligonucleotide stem is hybridized to the fourth overhang. In some embodiments, the plurality of oligonucleotide branches of the first oligonucleotide dendron, the plurality of oligonucleotide branches of the second oligonucleotide dendron, the plurality of oligonucleotide branches of the third oligonucleotide dendron, and the plurality of oligonucleotide branches of the fourth oligonucleotide dendron have identical nucleotide sequences. In further embodiments, the plurality of oligonucleotide branches of the first oligonucleotide dendron, the plurality of oligonucleotide branches of the second oligonucleotide dendron, the plurality of oligonucleotide branches of the third oligonucleotide dendron, and the plurality of oligonucleotide branches of the fourth oligonucleotide dendron have different nucleotide sequences. In still further embodiments, the plurality of oligonucleotide branches of the first oligonucleotide dendron and the plurality of oligonucleotide branches of the second oligonucleotide dendron have identical nucleotide sequences; the plurality of oligonucleotide branches of the third oligonucleotide dendron and the plurality of oligonucleotide branches of the fourth oligonucleotide dendron have identical nucleotide sequences, and the plurality of oligonucleotide branches of the first oligonucleotide dendron and the plurality of oligonucleotide branches of the second oligonucleotide dendron have nucleotide sequences that are different from nucleotide sequences of the plurality of oligonucleotide branches of the first oligonucleotide dendron and the plurality of oligonucleotide branches of the second oligonucleotide dendron. In some embodiments, in the target orientation the first overhang and the second overhang are oppositely disposed, and the third overhang and the fourth overhang are oppositely disposed. In some embodiments, the first overhang is about 4 to about 40 nucleotides in length. In some embodiments, the first overhang is about 18 nucleotides in length. In some embodiments, the second overhang is about 4 to about 40 nucleotides in length. In further embodiments, the second overhang is about 18 nucleotides in length. In some embodiments, the third overhang is about 4 to about 40 nucleotides in length. In some embodiments, the third overhang is about 18 nucleotides in length. In some embodiments, the fourth overhang is about 4 to about 40 nucleotides in length. In further embodiments, the fourth overhang is about 18 nucleotides in length. In some embodiments, the first oligonucleotide and the second oligonucleotide comprise DNA, RNA, a modified oligonucleotide, or a combination thereof. In some embodiments, the third oligonucleotide comprises DNA, RNA, a modified oligonucleotide, or a combination thereof. In some embodiments, the fourth oligonucleotide comprises DNA, RNA, a modified oligonucleotide, or a combination thereof. In some embodiments, the template core comprises a fifth oligonucleotide and hybridization of the first oligonucleotide, the second oligonucleotide, the third oligonucleotide, the fourth oligonucleotide, and the fifth oligonucleotide creates a fifth overhang, wherein the first overhang, the second overhang, the third overhang, the fourth overhang, and the fifth overhang are arranged in a target orientation, the oligonucleotide dendrimer further comprising a fifth oligonucleotide dendron comprising an oligonucleotide stem linked to a plurality of oligonucleotide branches, wherein the oligonucleotide stem is hybridized to the fifth overhang. In some embodiments, the template core comprises a sixth oligonucleotide and hybridization of the first oligonucleotide, the second oligonucleotide, the third oligonucleotide, the fourth oligonucleotide, the fifth oligonucleotide, and the sixth oligonucleotide creates a sixth overhang, wherein the first overhang, the second overhang, the third overhang, the fourth overhang, the fifth overhang, and the sixth overhang are arranged in a target orientation, the oligonucleotide dendrimer further comprising a sixth oligonucleotide dendron comprising an oligonucleotide stem linked to a plurality of oligonucleotide branches, wherein the oligonucleotide stem is hybridized to the sixth overhang. In some embodiments, the template core comprises a seventh oligonucleotide and hybridization of the first oligonucleotide, the second oligonucleotide, the third oligonucleotide, the fourth oligonucleotide, the fifth oligonucleotide, the sixth oligonucleotide, and the seventh oligonucleotide creates a seventh overhang, wherein the first overhang, the second overhang, the third overhang, the fourth overhang, the fifth overhang, the sixth overhang, and the seventh overhang are arranged in a target orientation, the oligonucleotide dendrimer further comprising a seventh oligonucleotide dendron comprising an oligonucleotide stem linked to a plurality of oligonucleotide branches, wherein the oligonucleotide stem is hybridized to the seventh overhang.

In some aspects, the disclosure provides a crystal comprising: (i) a plurality of oligonucleotide dendrimers, each oligonucleotide dendrimer comprising: a template core comprising first and second oligonucleotides hybridized to each other such that the template core has first and second overhangs, wherein the first and second overhangs are in a target orientation, a first oligonucleotide dendron comprising an oligonucleotide stem linked to a plurality of oligonucleotide branches, with the oligonucleotide stem being hybridized to the first overhang, and a second oligonucleotide dendron comprising an oligonucleotide stem linked to a plurality of oligonucleotide branches, with the oligonucleotide stem being hybridized to the second overhang; (ii) a plurality of first spherical nucleic acids (SNAs), each comprising a nanoparticle core and a plurality of oligonucleotides attached to the external surface of the nanoparticle core, wherein one or more oligonucleotides of the plurality of oligonucleotides of each of the first SNAs is hybridized to one or more oligonucleotide branches of the plurality of oligonucleotide branches of at least one first oligonucleotide dendron of the plurality of oligonucleotide dendrimers; and (iii) a plurality of second spherical nucleic acids (SNAs), each comprising a nanoparticle core and a plurality of oligonucleotides attached to the external surface of the nanoparticle core, wherein one or more oligonucleotides of the plurality of oligonucleotides of each of the second SNAs is hybridized to one or more oligonucleotide branches of the plurality of oligonucleotide branches of at least one second oligonucleotide dendron of the plurality of oligonucleotide dendrimers. In some embodiments, in the target orientation the first overhang and the second overhang are oppositely disposed. In some embodiments, a crystal of the disclosure further comprises a plurality of third spherical nucleic acids (SNAs), each comprising a nanoparticle core and a plurality of oligonucleotides attached to the external surface of the nanoparticle core, wherein: the template core of each of the plurality of oligonucleotide dendrimers comprises a third oligonucleotide that is hybridized to the first oligonucleotide and the second oligonucleotide such that the template core comprises a third overhang, wherein the first overhang, the second overhang, and the third overhang are in the target orientation, and each of the plurality of dendrimers further comprises a third oligonucleotide dendron comprising an oligonucleotide stem linked to a plurality of oligonucleotide branches, with the oligonucleotide stem being hybridized to the third overhang, and one or more oligonucleotides of the plurality of oligonucleotides of each of the third SNAs is hybridized to one or more oligonucleotide branches of the plurality of oligonucleotide branches of at least one third oligonucleotide dendron. In some embodiments, in the target orientation the first overhang, the second overhang, and the third overhang are in a radial orientation. In some embodiments, a crystal of the disclosure further comprises a plurality of fourth spherical nucleic acids (SNAs) comprising a nanoparticle core and a plurality of oligonucleotides attached to the external surface of the nanoparticle core, wherein: the template core of each of the plurality of oligonucleotide dendrimers comprises a fourth oligonucleotide and hybridization of the first oligonucleotide, the second oligonucleotide, the third oligonucleotide, and the fourth oligonucleotide results in a fourth overhang, wherein the first overhang, the second overhang, the third overhang, and the fourth overhang are in the target orientation, and each of the plurality of dendrimers further comprises a fourth oligonucleotide dendron comprising an oligonucleotide stem linked to a plurality of oligonucleotide branches, with the oligonucleotide stem being hybridized to the fourth overhang, and one or more oligonucleotides of the plurality of oligonucleotides of each of the fourth SNAs is hybridized to one or more oligonucleotide branches of the plurality of oligonucleotide branches of the fourth oligonucleotide dendron. In some embodiments, in the target orientation the first overhang and the second overhang are oppositely disposed, and the third overhang and the fourth overhang are oppositely disposed. In some embodiments, a crystal of the disclosure further comprises a plurality of fifth spherical nucleic acids (SNAs) comprising a nanoparticle core and a plurality of oligonucleotides attached to the external surface of the nanoparticle core, wherein: the template core of each of the plurality of oligonucleotide dendrimers comprises a fifth oligonucleotide and hybridization of the first oligonucleotide, the second oligonucleotide, third oligonucleotide, fourth oligonucleotide, and fifth oligonucleotide results in a fifth overhang, wherein the first overhang, the second overhang, the third overhang, the fourth overhang, and the fifth overhang are in the target orientation, and each of the plurality of dendrimers further comprises a fifth oligonucleotide dendron comprising an oligonucleotide stem linked to a plurality of oligonucleotide branches, with the oligonucleotide stem being hybridized to the fifth overhang, and one or more oligonucleotides of the plurality of oligonucleotides of each of the fifth SNAs is hybridized to one or more oligonucleotide branches of the plurality of oligonucleotide branches of the fifth oligonucleotide dendron. In some embodiments, a crystal of the disclosure further comprises a plurality of sixth spherical nucleic acids (SNAs) comprising a nanoparticle core and a plurality of oligonucleotides attached to the external surface of the nanoparticle core, wherein: the template core of each of the plurality of oligonucleotide dendrimers comprises a sixth oligonucleotide and hybridization of the first oligonucleotide, the second oligonucleotide, the third oligonucleotide, the fourth oligonucleotide, the fifth oligonucleotide, and the sixth oligonucleotide results in a sixth overhang, wherein the first overhang, the second overhang, the third overhang, the fourth overhang, the fifth overhang, and the sixth overhang are in the target orientation, and each of the plurality of dendrimers further comprises a sixth oligonucleotide dendron comprising an oligonucleotide stem linked to a plurality of oligonucleotide branches, with the oligonucleotide stem being hybridized to the sixth overhang, and one or more oligonucleotides of the plurality of oligonucleotides of each of the sixth SNAs is hybridized to one or more oligonucleotide branches of the plurality of oligonucleotide branches of the sixth oligonucleotide dendron. In some embodiments, a crystal of the disclosure further comprises a plurality of seventh spherical nucleic acids (SNAs) comprising a nanoparticle core and a plurality of oligonucleotides attached to the external surface of the nanoparticle core, wherein: the template core of each of the plurality of oligonucleotide dendrimers comprises a seventh oligonucleotide and hybridization of the first oligonucleotide, the second oligonucleotide, the third oligonucleotide, the fourth oligonucleotide, the fifth oligonucleotide, the sixth oligonucleotide, and the seventh oligonucleotide results in a seventh overhang, wherein the first overhang, the second overhang, the third overhang, the fourth overhang, the fifth overhang, the sixth overhang, and the seventh overhang are in the target orientation, and each of the plurality of dendrimers further comprises a seventh oligonucleotide dendron comprising an oligonucleotide stem linked to a plurality of oligonucleotide branches, with the oligonucleotide stem being hybridized to the seventh overhang, and one or more oligonucleotides of the plurality of oligonucleotides of each of the seventh SNAs is hybridized to one or more oligonucleotide branches of the plurality of oligonucleotide branches of the seventh oligonucleotide dendron. In some embodiments, the first oligonucleotide dendron, the second oligonucleotide dendron, the third oligonucleotide dendron, the fourth oligonucleotide dendron, the fifth oligonucleotide dendron, the sixth oligonucleotide dendron, and/or the seventh oligonucleotide dendron is a DNA dendron, a RNA dendron, a modified oligonucleotide dendron, or a combination thereof. In some embodiments, the first oligonucleotide dendron, the second oligonucleotide dendron, the third oligonucleotide dendron, and/or the fourth oligonucleotide dendron of an oligonucleotide dendrimer or a crystal of the disclosure each comprises about 2 to about 27 oligonucleotide branches. In further embodiments, the first oligonucleotide dendron, the second oligonucleotide dendron, the third oligonucleotide dendron, and/or the fourth oligonucleotide dendron of an oligonucleotide dendrimer or a crystal of the disclosure each comprises 6 branches. In further embodiments, the first oligonucleotide dendron, the second oligonucleotide dendron, the third oligonucleotide dendron, and/or the fourth oligonucleotide dendron of an oligonucleotide dendrimer or a crystal of the disclosure each comprises 9 branches. In some embodiments, the first oligonucleotide dendron, the second oligonucleotide dendron, the third oligonucleotide dendron, and/or the fourth oligonucleotide dendron of an oligonucleotide dendrimer or a crystal of the disclosure is a DNA dendron, a RNA dendron, a modified oligonucleotide dendron, or a combination thereof.

In some aspects, the disclosure provides a method of forming a crystal, comprising: contacting a plurality of oligonucleotide dendrimers with a plurality of first and second spherical nucleic acids (SNAs) such that the plurality of oligonucleotide dendrimers and the plurality of first and second SNAs hybridize to thereby arrange the first and second SNAS to form the crystal, wherein: the plurality of oligonucleotide dendrimers each comprises: a template core comprising first and second oligonucleotides hybridized to each other such that the template core has first and second overhangs, wherein the first and second overhangs are in a target orientation, a first oligonucleotide dendron comprising an oligonucleotide stem linked to a plurality of oligonucleotide branches, with the oligonucleotide stem being hybridized to the first overhang, and a second oligonucleotide dendron comprising an oligonucleotide stem linked to a plurality of oligonucleotide branches, with the oligonucleotide stem being hybridized to the second overhang, each of the plurality of first SNAs comprises a nanoparticle core and a plurality of oligonucleotides attached to an external surface of the nanoparticle core, each of the plurality of second SNAs comprises a nanoparticle core and a plurality of oligonucleotides attached to an external surface of the nanoparticle core, upon contact: one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, and one or more oligonucleotides of the plurality of oligonucleotides of each of the second SNAs is hybridizes to one or more oligonucleotide branches of the plurality of oligonucleotide branches of at least one second oligonucleotide dendron of the plurality of oligonucleotide dendrimers. In some embodiments, in the target orientation the first overhang and the second overhang are oppositely disposed.

In further aspects, the disclosure provides a method of forming a crystal, comprising: contacting a plurality of oligonucleotide dendrimers with a plurality of first, second, and third spherical nucleic acids (SNAs) such that the plurality of oligonucleotide dendrimers and the plurality of first, second, and third SNAs hybridize to thereby arrange the first, second, and third SNAs to form the crystal, wherein: the plurality of oligonucleotide dendrimers each comprises: a template core comprising first, second, and third oligonucleotides hybridized to each other such that the template core has first, second, and third overhangs, wherein the first overhang, the second overhang, and the third overhang are in a target orientation, a first oligonucleotide dendron comprising an oligonucleotide stem linked to a plurality of oligonucleotide branches, with the oligonucleotide stem being hybridized to the first overhang, a second oligonucleotide dendron comprising an oligonucleotide stem linked to a plurality of oligonucleotide branches, with the oligonucleotide stem being hybridized to the second overhang, and a third oligonucleotide dendron comprising an oligonucleotide stem linked to a plurality of oligonucleotide branches, with the oligonucleotide stem being hybridized to the third overhang, each of the plurality of first SNAs comprises a nanoparticle core and a plurality of oligonucleotides attached to an external surface of the nanoparticle core, each of the plurality of second SNAs comprises a nanoparticle core and a plurality of oligonucleotides attached to an external surface of the nanoparticle core, each of the plurality of third SNAs comprises a nanoparticle core and a plurality of oligonucleotides attached to an external surface of the nanoparticle core, upon contact: one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers, and one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers. In some embodiments, in the target orientation the first overhang, the second overhang, and the third overhang are in a radial orientation.

In some aspects, the disclosure provides a method of forming a crystal, comprising: contacting a plurality of oligonucleotide dendrimers with a plurality of first, second, third, and fourth spherical nucleic acids (SNAs) such that the plurality of oligonucleotide dendrimers and the plurality of first, second, third, and fourth SNAs hybridize to thereby arrange the first, second, third, and fourth SNAs to form the crystal, wherein: the plurality of oligonucleotide dendrimers each comprises: a template core comprising first, second, third, and fourth oligonucleotides hybridized to each other such that the template core has first, second, third, and fourth overhangs, wherein the first overhang, the second overhang, the third overhang, and the fourth overhang are in a target orientation, a first oligonucleotide dendron comprising an oligonucleotide stem linked to a plurality of oligonucleotide branches, with the oligonucleotide stem being hybridized to the first overhang, a second oligonucleotide dendron comprising an oligonucleotide stem linked to a plurality of oligonucleotide branches, with the oligonucleotide stem being hybridized to the second overhang, and a third oligonucleotide dendron comprising an oligonucleotide stem linked to a plurality of oligonucleotide branches, with the oligonucleotide stem being hybridized to the third overhang, a fourth oligonucleotide dendron comprising an oligonucleotide stem linked to a plurality of oligonucleotide branches, with the oligonucleotide stem being hybridized to the fourth overhang, each of the plurality of first SNAs comprises a nanoparticle core and a plurality of oligonucleotides attached to an external surface of the nanoparticle core, each of the plurality of second SNAs comprises a nanoparticle core and a plurality of oligonucleotides attached to an external surface of the nanoparticle core, each of the plurality of third SNAs comprises a nanoparticle core and a plurality of oligonucleotides attached to an external surface of the nanoparticle core, each of the plurality of fourth SNAs comprises a nanoparticle core and a plurality of oligonucleotides attached to an external surface of the nanoparticle core, upon contact: one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers, one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers, and one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers. In some embodiments, in the target orientation the first overhang and the second overhang are oppositely disposed, and the third overhang and the fourth overhang are oppositely disposed.

In further aspects, the disclosure provides a method of forming a crystal, comprising: contacting a plurality of oligonucleotide dendrimers with a plurality of first, second, third, fourth, and fifth spherical nucleic acids (SNAs) such that the plurality of oligonucleotide dendrimers and the plurality of first, second, third, fourth, and fifth SNAs hybridize to thereby arrange the first, second, third, fourth, and fifth SNAs to form the crystal, wherein: the plurality of oligonucleotide dendrimers each comprises: a template core comprising first, second, third, fourth, and fifth oligonucleotides hybridized to each other such that the template core has first, second, third, fourth, and fifth overhangs, wherein the first overhang, the second overhang, the third overhang, the fourth overhang, and the fifth overhang are in a target orientation, a first oligonucleotide dendron comprising an oligonucleotide stem linked to a plurality of oligonucleotide branches, with the oligonucleotide stem being hybridized to the first overhang, a second oligonucleotide dendron comprising an oligonucleotide stem linked to a plurality of oligonucleotide branches, with the oligonucleotide stem being hybridized to the second overhang, and a third oligonucleotide dendron comprising an oligonucleotide stem linked to a plurality of oligonucleotide branches, with the oligonucleotide stem being hybridized to the third overhang, a fourth oligonucleotide dendron comprising an oligonucleotide stem linked to a plurality of oligonucleotide branches, with the oligonucleotide stem being hybridized to the fourth overhang, a fifth oligonucleotide dendron comprising an oligonucleotide stem linked to a plurality of oligonucleotide branches, with the oligonucleotide stem being hybridized to the fifth overhang, each of the plurality of first SNAs comprises a nanoparticle core and a plurality of oligonucleotides attached to an external surface of the nanoparticle core, each of the plurality of second SNAs comprises a nanoparticle core and a plurality of oligonucleotides attached to an external surface of the nanoparticle core, each of the plurality of third SNAs comprises a nanoparticle core and a plurality of oligonucleotides attached to an external surface of the nanoparticle core, each of the plurality of fourth SNAs comprises a nanoparticle core and a plurality of oligonucleotides attached to an external surface of the nanoparticle core, each of the plurality of fifth SNAs comprises a nanoparticle core and a plurality of oligonucleotides attached to an external surface of the nanoparticle core, upon contact: one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers, one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers, one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers, and one or more oligonucleotides of the plurality of oligonucleotides of each of the fifth SNAs hybridizes to one or more oligonucleotide branches of the plurality of oligonucleotide branches of at least one fifth oligonucleotide dendron of the plurality of oligonucleotide dendrimers.

In some aspects, the disclosure provides a method of forming a crystal, comprising: contacting a plurality of oligonucleotide dendrimers with a plurality of first, second, third, fourth, fifth, and sixth spherical nucleic acids (SNAs) such that the plurality of oligonucleotide dendrimers and the plurality of first, second, third, fourth, fifth, and sixth SNAs hybridize to thereby arrange the first, second, third, fourth, fifth, and sixth SNAs to form the crystal, wherein: the plurality of oligonucleotide dendrimers each comprises: a template core comprising first, second, third, fourth, fifth, and sixth oligonucleotides hybridized to each other such that the template core has first, second, third, fourth, fifth, and sixth overhangs, wherein the first overhang, the second overhang, the third overhang, the fourth overhang, the fifth overhang, and the sixth overhang are in a target orientation, a first oligonucleotide dendron comprising an oligonucleotide stem linked to a plurality of oligonucleotide branches, with the oligonucleotide stem being hybridized to the first overhang, a second oligonucleotide dendron comprising an oligonucleotide stem linked to a plurality of oligonucleotide branches, with the oligonucleotide stem being hybridized to the second overhang, and a third oligonucleotide dendron comprising an oligonucleotide stem linked to a plurality of oligonucleotide branches, with the oligonucleotide stem being hybridized to the third overhang, a fourth oligonucleotide dendron comprising an oligonucleotide stem linked to a plurality of oligonucleotide branches, with the oligonucleotide stem being hybridized to the fourth overhang, a fifth oligonucleotide dendron comprising an oligonucleotide stem linked to a plurality of oligonucleotide branches, with the oligonucleotide stem being hybridized to the fifth overhang, a sixth oligonucleotide dendron comprising an oligonucleotide stem linked to a plurality of oligonucleotide branches, with the oligonucleotide stem being hybridized to the sixth overhang, each of the plurality of first SNAs comprises a nanoparticle core and a plurality of oligonucleotides attached to an external surface of the nanoparticle core, each of the plurality of second SNAs comprises a nanoparticle core and a plurality of oligonucleotides attached to an external surface of the nanoparticle core, each of the plurality of third SNAs comprises a nanoparticle core and a plurality of oligonucleotides attached to an external surface of the nanoparticle core, each of the plurality of fourth SNAs comprises a nanoparticle core and a plurality of oligonucleotides attached to an external surface of the nanoparticle core, each of the plurality of fifth SNAs comprises a nanoparticle core and a plurality of oligonucleotides attached to an external surface of the nanoparticle core, each of the plurality of sixth SNAs comprises a nanoparticle core and a plurality of oligonucleotides attached to an external surface of the nanoparticle core, upon contact: one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers, one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers, one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers, one or more oligonucleotides of the plurality of oligonucleotides of each of the fifth SNAs hybridizes to one or more oligonucleotide branches of the plurality of oligonucleotide branches of at least one fifth oligonucleotide dendron of the plurality of oligonucleotide dendrimers, and one or more oligonucleotides of the plurality of oligonucleotides of each of the sixth SNAs hybridizes to one or more oligonucleotide branches of the plurality of oligonucleotide branches of at least one sixth oligonucleotide dendron of the plurality of oligonucleotide dendrimers.

In some aspects, the disclosure provides a method of forming a crystal, comprising: contacting a plurality of oligonucleotide dendrimers with a plurality of first, second, third, fourth, fifth, sixth, and seventh spherical nucleic acids (SNAs) such that the plurality of oligonucleotide dendrimers and the plurality of first, second, third, fourth, fifth, sixth, and seventh SNAs hybridize to thereby arrange the first, second, third, fourth, fifth, sixth, and seventh SNAs to form the crystal, wherein: the plurality of oligonucleotide dendrimers each comprises: a template core comprising first, second, third, fourth, fifth, sixth, and seventh oligonucleotides hybridized to each other such that the template core has first, second, third, fourth, fifth, sixth, and seventh overhangs, wherein the first overhang, the second overhang, the third overhang, the fourth overhang, the fifth overhang, the sixth overhang, and the seventh are in a target orientation, a first oligonucleotide dendron comprising an oligonucleotide stem linked to a plurality of oligonucleotide branches, with the oligonucleotide stem being hybridized to the first overhang, a second oligonucleotide dendron comprising an oligonucleotide stem linked to a plurality of oligonucleotide branches, with the oligonucleotide stem being hybridized to the second overhang, and a third oligonucleotide dendron comprising an oligonucleotide stem linked to a plurality of oligonucleotide branches, with the oligonucleotide stem being hybridized to the third overhang, a fourth oligonucleotide dendron comprising an oligonucleotide stem linked to a plurality of oligonucleotide branches, with the oligonucleotide stem being hybridized to the fourth overhang, a fifth oligonucleotide dendron comprising an oligonucleotide stem linked to a plurality of oligonucleotide branches, with the oligonucleotide stem being hybridized to the fifth overhang, a sixth oligonucleotide dendron comprising an oligonucleotide stem linked to a plurality of oligonucleotide branches, with the oligonucleotide stem being hybridized to the sixth overhang, a seventh oligonucleotide dendron comprising an oligonucleotide stem linked to a plurality of oligonucleotide branches, with the oligonucleotide stem being hybridized to the seventh overhang, each of the plurality of first SNAs comprises a nanoparticle core and a plurality of oligonucleotides attached to an external surface of the nanoparticle core, each of the plurality of second SNAs comprises a nanoparticle core and a plurality of oligonucleotides attached to an external surface of the nanoparticle core, each of the plurality of third SNAs comprises a nanoparticle core and a plurality of oligonucleotides attached to an external surface of the nanoparticle core, each of the plurality of fourth SNAs comprises a nanoparticle core and a plurality of oligonucleotides attached to an external surface of the nanoparticle core, each of the plurality of fifth SNAs comprises a nanoparticle core and a plurality of oligonucleotides attached to an external surface of the nanoparticle core, each of the plurality of sixth SNAs comprises a nanoparticle core and a plurality of oligonucleotides attached to an external surface of the nanoparticle core, each of the plurality of seventh SNAs comprises a nanoparticle core and a plurality of oligonucleotides attached to an external surface of the nanoparticle core, upon contact: one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers, one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers, one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers, one or more oligonucleotides of the plurality of oligonucleotides of each of the fifth SNAs hybridizes to one or more oligonucleotide branches of the plurality of oligonucleotide branches of at least one fifth oligonucleotide dendron of the plurality of oligonucleotide dendrimers, and one or more oligonucleotides of the plurality of oligonucleotides of each of the sixth SNAs hybridizes to one or more oligonucleotide branches of the plurality of oligonucleotide branches of at least one sixth oligonucleotide dendron of the plurality of oligonucleotide dendrimers, and one or more oligonucleotides of the plurality of oligonucleotides of each of the seventh SNAs hybridizes to one or more oligonucleotide branches of the plurality of oligonucleotide branches of at least one seventh oligonucleotide dendron of the plurality of oligonucleotide dendrimers. In some embodiments, each of the plurality of oligonucleotide dendrimers is an oligonucleotide dendrimer of the disclosure. In some embodiments, the crystal is a crystal as described herein. In some embodiments, a method of the disclosure further comprises contacting the crystal with a chemical input, thereby resulting in at least partial disassembly of the crystal. In further embodiments, the chemical input is toe-hold mediated strand displacement, a light responsive chemistry, a pH responsive chemistry, or a combination thereof.

### BRIEF DESCRIPTION OF THE DRAWINGS

FIG. 1 shows that DNA dendrons have the potential to program unique, orthogonal interactions in colloidal crystals, a capability that has long been elusive in this field. Individual DNA dendrons can be designed to contain orthogonal sticky ends (branches) that hybridize with different strands on DNA-functionalized nanoparticles (NPs) (top). The stems of each of these dendrons are designed to form DNA junctions with one another (middle). As a result, Electron Equivalents (EEs) and Programmable Atom Equivalents (PAEs) with organized clusters of orthogonal sticky ends can then direct the assembly of multiple NP components into a single, ordered, crystalline assembly (bottom).

FIG. 2 shows that DNA dendrons are purified by denaturing PAGE. To form junctions, the DNA dendrons were annealed in 0.5 M NaCl at 95° C. for 10 minutes and cooled from 95° C. to 20° C. over 1 hour. Resultant junctions were characterized by native PAGE as shown for the 2-way junction (a), the 3-way junction (b), and the 4-way junction (c).

FIG. 3 shows: (a) DNA Junction melting temperatures were determined by measuring the fluorescence signal of the intercalating dye, SYBR Green, as the junctions were heated and de-hybridized. The derivative of that melting curve indicates the two-way junction has a Tₘ of 77.4° C., the three-way junction has a Tₘ of 58.4° C., and the four-way junction has a Tₘ of 53.6° C. (b) The size of each junction was determined by DLS. The junctions have hydrodynamic diameters of 4.3 nm, 6.1 nm, and 8.2 nm, for the two-, three-, and four-way junctions, respectively.

FIG. 4 shows that DNA functionalized 10 nm gold nanoparticles (AuNPs) were assembled with each of the DNA junctions. The thermostability of the resultant assemblies was measured by a UV-vis melt experiment. A characteristic increase in the melting temperature was observed as the valency and size of the DNA junction PAEs (DJ PAEs) increased. While the melting temperature changed, the crystal structure remained constant due to the vast size difference between the Au PAEs and the DJ PAEs, forming an FCC crystal. Thus, the experiments revealed increasing Tₘ with increasing DJ valency.

FIG. 5 shows that DNA functionalized 10 nm AuNPs were assembled with each of the DNA junctions. While the melting temperature changes (see FIG. 4), the crystal structure remains constant due to the vast size difference between the Au PAEs and the DJ PAEs, forming an FCC crystal. Thus, the experiments showed that DJ PAE Size favors FCC crystal structure formation.

FIG. 6 depicts that the oligonucleotide (e.g., DNA) dendron design described herein allows greater programmability over the DJ PAE architecture. Linear DNA strands were used to form a template core in the form of a DNA junction. In some embodiments, the templates have 18 base overhangs to which the stems of the dendrons can hybridize. This approach provides more design handles that allows us to more easily tune the thermostabilities and organizations of resultant assemblies. Larger DJ PAEs provide greater tunability over assembly outcomes.

FIG. 7 shows melt data of the DNA junction templates that were collected, showing an expected decrease in melting temperature as the junction valency was increased. The melt data matched the simulated Tₘ values of the junction cores.

FIG. 8 shows results of experiments in which larger DJ PAEs were assembled with 10 nm AuNP PAEs to form colloidal crystals. Melting experiments revealed an expected increase in colloidal crystal thermostability as the junction core increased in valency. This was expected because the 4-way DJ PAE would have significantly more sticky ends, thereby forming more favorable interactions with the AuNP PAEs. Thus, the melt data of colloidal crystals further confirmed DJ PAE formation.

FIG. 9 depicts an experimental design for determining if two types of nanoparticles can be incorporated into a single colloidal crystal using the technology described herein. Both NP types were present in solution. (Left) Only one dendron was present so only one particle type can assemble. (Middle) Only the other dendron was present so the other NP type could assemble. (Right) Both dendrons were present and form a DNA junction so that both particles can be assembled into a single colloidal crystal. Qualitative data supports that both nanoparticles are assembled into colloidal crystals.

FIG. 10 shows results of melting experiments that were conducted on the assemblies from FIG. 9. In the samples that only contained a single dendron, the melting temperature was around 26 C. These results matched previous data for single dendrons used in NP assembly. However, when both dendrons were present and form a DNA junction, a single, significantly higher melting transition was observed, indicating that both particles were incorporated into a single, more stable, crystal structure. Thus, melt data showed incorporation of both nanoparticles in a single colloidal crystal.

FIG. 11 depicts an experimental design for determining if two types of nanoparticles can be incorporated into a single colloidal crystal using the larger DJ PAE approach. Both NP types were present in solution. (Left) Only one dendron was present so only one particle type can assemble. (Middle-Left)) Only the other dendron was present so the other NP type could assemble. (Middle-Right) Both dendrons were present but no DNA junction template was provided. This means that the dendrons would not be able to form into a DJ PAE and therefore should behave separately in solution. (Right) Both dendrons and the DNA junction template were present. Therefore, the entire DJ PAE could form and ideally both particles will be incorporate into a single crystal. Qualitative data supports that both nanoparticles are incorporated in the colloidal crystal.

FIG. 12 shows results of melting experiments that were conducted on the assemblies from FIG. 11. Single dendron containing assemblies had low melting temperatures that matched previous results. Interestingly, when both dendrons were present but had no DNA junction core to connect them (black trace), two separate melting transitions formed: one that matched the melting temperature of the orange trace, and one that matched the melting temperature of the blue trace. This indicated that without the DNA junction core, two separate colloidal crystals formed with their own distinct thermostabilities. Finally, when all components were combined to form the complete DJ PAE, a significantly higher, single melting transition was observed. This data supported that only when all components are present do the colloidal crystals contain both nanoparticle types within a single colloidal crystal. Thus, melt data supported that both nanoparticles were incorporated in the colloidal crystal.

FIG. 13 shows results of a similar melting experiment that was conducted on assemblies that were formed containing three unique AuNP PAEs. The assembly that contained all three dendrons, but no junction core experienced three separate melting transitions. This indicates that while assemblies formed under these conditions, each unique particle type formed a separate colloidal crystal (red trace). When all three dendrons and the junction core were present in the assembly solution, the fully composed DJ PAE could form. As a result, a single melting transition was observed, indicating that all three particle types were able incorporated into a single colloidal crystal (black trace). Thus, melt data supported that three unique nanoparticles were incorporated in the colloidal crystal.

### DETAILED DESCRIPTION

Nature has developed powerful techniques to program sequence specific interactions between multiple building blocks. Such multicomponent assemblies are responsible for important biological processes and properties. Nevertheless, methods to synthetically produce multicomponent assemblies are limited in their programmability and complexity, stifling the development of new materials. The present disclosure describes the synthesis and utilization of oligonucleotide (e.g., DNA) dendrimers that are capable of programming the formation of multicomponent assemblies in the form of colloidal crystals. By combining principles from oligonucleotide (e.g., DNA)-mediated nanoparticle assembly and oligonucleotide (e.g., DNA) nanotechnology, multicomponent colloidal crystals with unprecedented control and complexity are provided herein. Individual oligonucleotide (e.g., DNA) dendrimers comprise multiple oligonucleotide (e.g., DNA) dendrons, each containing a defined number of single stranded oligonucleotides (i.e., sticky ends) that can be either identical or orthogonal to those of other dendrons in the structure. These oligonucleotide (e.g., DNA) dendrimers assemble with complementary oligonucleotide (e.g., DNA)-functionalized nanoparticles to yield colloidal crystals with novel structural precision. It is demonstrated herein that by tuning the oligonucleotide (e.g., DNA) dendrimer design, colloidal crystals containing two, three, and four unique components are synthesized such that the location of each component within the unit cell is discrete and known. Moreover, the lattice parameters, crystal structures, and component crystal locations can all be programmed by systematically changing the size and valency of the oligonucleotide (e.g., DNA) dendrimers. Further, it is demonstrated herein that through precise oligonucleotide (e.g., DNA) design, responsive, multicomponent colloidal crystals can be synthesized, such that a specific component can be removed from a colloidal crystal in response to a chemical input. The present disclosure therefore provides a widely applicable and easily adaptable platform to synthesize multicomponent nanoparticle assemblies regardless of component size, shape, and composition. The present disclosure also lays the foundation to develop complex, multifunctional materials that are capable of electron/energy transfer, catalytic/enzymatic cascades, and other emergent optical, electronic, and magnetic properties, thereby expanding capabilities in materials design.

### Methods of Forming Multicomponent Assemblies

Nanoparticle assembly is an increasingly desirable capability for the development of modern and next-generation materials. The approach to programmable assembly provided herein provides a robust, safe, and sustainable method for organizing nanoparticles into crystalline, ordered assemblies independent of their size, shape, and composition. Furthermore, the methods of the disclosure are highly modular and reconfigurable that enables access to a vast array of crystal structures, lattice parameters, and thermostabilities.

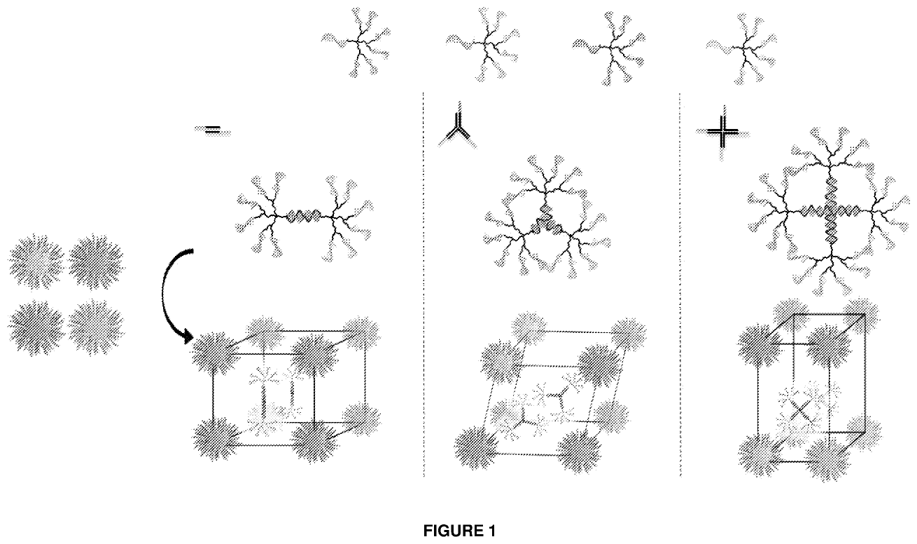

In some aspects, the disclosure provides methods of forming multicomponent assemblies, such as crystalline assemblies, using oligonucleotides. In general, the methods comprise associating a plurality of oligonucleotide dendrimers with a plurality of SNAs via hybridization interactions (see, e.g., FIG. 1). Each of the oligonucleotide dendrimers is produced by hybridizing at least two oligonucleotide dendrons to a template core, wherein the template core comprises at least two overhangs in a target orientation. In some embodiments, the template core comprises at least two oligonucleotides that are hybridized to each other to create at least two overhangs in a target orientation. As used herein, a “target orientation” is an orientation that a given number of overhangs adopts following hybridization of oligonucleotides. The target orientation can take on different conformations. In every case, the target orientation will depend on the number of oligonucleotides used to generate the template core. For example and without limitation, when three oligonucleotides are used to generate a template core then the template core will have three separate overhang “arms”; when four oligonucleotides are used to generate a template core then the template core will have four separate overhang “arms”. In general, when the template core comprises two oligonucleotides, then the template core will have a first and a second overhang and the first and second overhangs will be oppositely disposed. In general, when the template core comprises three oligonucleotides, then the template core will have a first, second, and third overhang and the first, second, and third overhangs will be in a general radial (e.g., a Y-shaped) orientation. In general, when the template core comprises four oligonucleotides, then the template core will have a first, second, third, and fourth overhang and the first overhang and the second overhang are oppositely disposed, and the third overhang and the fourth overhang are oppositely disposed. Thus, in various embodiments, the template core comprises two, three, four, five, six, seven, or more oligonucleotides that are hybridized to each other to create at least two, three, four, five, six, seven, or more overhangs, respectively. In some embodiments, the template core comprises a small molecule core that has oligonucleotides attached to it (see, e.g., Cheng et al., J. Am. Chem. Soc. 2021, 143, 4, 1752-1757 and Cheng, Distler et al., J. Am. Chem. Soc. 2021, 143, 41, 17170-17179) to create at least two, three, four, five, six, or seven overhangs in a target orientation. Each oligonucleotide dendron comprises an oligonucleotide stem linked to a plurality of oligonucleotide branches, wherein the oligonucleotide stem hybridizes to an overhang of the template core. Each of the SNAs comprises a nanoparticle core and a plurality of oligonucleotides that is attached to the external surface of the nanoparticle core. The oligonucleotide dendron and the SNA are designed such that an oligonucleotide branch of the oligonucleotide dendron can hybridize to an oligonucleotide in the plurality of oligonucleotides that are attached to the external surface of the nanoparticle core of the SNA. Thus, by varying the structure of the template core (and therefore the structure of the oligonucleotide dendrimer), formation of different multicomponent assemblies is achieved. In various embodiments, the oligonucleotide stem, the oligonucleotide branches, the overhangs, and/or the oligonucleotides attached to the nanoparticle core of each SNA are single stranded. In some embodiments, one or more of the oligonucleotide stem, the oligonucleotide branches, and the oligonucleotides attached to the nanoparticle core of each SNA are double stranded. In any of the embodiments or aspects of the disclosure, an oligonucleotide dendrimer comprises double stranded portions (i.e., the portion of the dendrimer in which oligonucleotide stems are hybridized to the template core) and single stranded portions (i.e., the oligonucleotide branches). In some embodiments, a SNA comprises single stranded and double stranded oligonucleotides in the plurality of oligonucleotides that are attached to the external surface of the nanoparticle core. In some embodiments, an oligonucleotide dendron comprises a plurality of oligonucleotide branches in which at least two of the oligonucleotide branches have different nucleotide sequences. In various embodiments, the template cores and the multicomponent assemblies are formed through annealing and slow cooling.

In any of the aspects or embodiments of the disclosure, a template core is formed by hybridizing two, three, four, five, six, seven, or more single stranded oligonucleotides to each other, wherein the hybridizing results in double stranded portions as well as two, three, four, five, six, seven, or more single stranded overhangs, respectively, arranged in a target orientation. See, e.g., FIG. 1. In any of the aspects or embodiments of the disclosure, an oligonucleotide dendron comprises a single stranded oligonucleotide stem and a plurality of single stranded oligonucleotide branches, and the single stranded oligonucleotide stem from at least two oligonucleotide dendrons individually hybridize to a single stranded overhang of a template core to create an oligonucleotide dendrimer. In any of the aspects or embodiments of the disclosure, at least two SNA populations are formed, wherein each SNA of each population comprises a nanoparticle core and a plurality of single stranded oligonucleotides that is attached to the external surface of the nanoparticle core, and a single stranded oligonucleotide branch of an oligonucleotide dendrimer hybridizes to an oligonucleotide in the plurality of oligonucleotides of a SNA. In any of the aspects or embodiments of the disclosure, a multicomponent assembly is formed by contacting a plurality of oligonucleotide dendrimers to a plurality of SNAs.

In some embodiments, a template core comprises two oligonucleotides that are hybridized to each other such that the template core comprises a first overhang and a second overhang, wherein the first overhang and the second overhang are oppositely disposed. The template core is then associated with a first and a second oligonucleotide dendron, each oligonucleotide dendron comprising an oligonucleotide stem linked to a plurality of oligonucleotide branches. The oligonucleotide stem of the first oligonucleotide dendron hybridizes to the first overhang of the template core, and the oligonucleotide stem of the second oligonucleotide dendron hybridizes to the second overhang of the template core to create an oligonucleotide dendrimer. A plurality of the oligonucleotide dendrimers is contacted with a plurality of first SNAs and second SNAs, each SNA comprising a nanoparticle core and a plurality of oligonucleotides attached to the external surface of the nanoparticle core, such that one or more oligonucleotide branches of a first oligonucleotide dendron hybridize to the plurality of oligonucleotides of the first SNA, and one or more oligonucleotide branches of a second oligonucleotide dendron hybridize to the plurality of oligonucleotides of the second SNA to create a multicomponent assembly. In some embodiments, each oligonucleotide branch of the first oligonucleotide dendron has the same nucleotide sequence, and each oligonucleotide branch of the second oligonucleotide dendron has the same nucleotide sequence, and the nucleotide sequences of the oligonucleotide branches of the first dendron are different than the nucleotide sequences of the oligonucleotide branches of the second dendron. In some embodiments, each oligonucleotide branch of the first oligonucleotide dendron has the same nucleotide sequence, and each oligonucleotide branch of the second oligonucleotide dendron has the same nucleotide sequence, and the nucleotide sequences of the oligonucleotide branches of the first dendron are the same as the nucleotide sequences of the oligonucleotide branches of the second dendron. In some embodiments, the first oligonucleotide dendron and/or the second oligonucleotide dendron comprise a plurality of oligonucleotide branches in which at least two of the oligonucleotide branches have different nucleotide sequences.

In some embodiments, a template core comprises three oligonucleotides that are hybridized to each other such that the template core comprises a first overhang, a second overhang, and a third overhang, wherein the first overhang, the second overhang, and the third overhang are in a Y-shaped orientation. The template core is then associated with a first, a second, and a third oligonucleotide dendron, each oligonucleotide dendron comprising an oligonucleotide stem linked to a plurality of oligonucleotide branches. The oligonucleotide stem of the first oligonucleotide dendron hybridizes to the first overhang of the template core, the oligonucleotide stem of the second oligonucleotide dendron hybridizes to the second overhang of the template core, and the oligonucleotide stem of the third oligonucleotide dendron hybridizes to the third overhang of the template core to create an oligonucleotide dendrimer. A plurality of the oligonucleotide dendrimers is contacted with a plurality of first SNAs, second SNAs, and third SNAs, each SNA comprising a nanoparticle core and a plurality of oligonucleotides attached to the external surface of the nanoparticle core, such that one or more oligonucleotide branches of a first oligonucleotide dendron hybridize to the plurality of oligonucleotides of the first SNA, one or more oligonucleotide branches of a second oligonucleotide dendron hybridize to the plurality of oligonucleotides of the second SNA, and one or more oligonucleotide branches of a third oligonucleotide dendron hybridize to the plurality of oligonucleotides of the third SNA to create a multicomponent assembly. In some embodiments, each oligonucleotide branch of the first oligonucleotide dendron has the same nucleotide sequence, each oligonucleotide branch of the second oligonucleotide dendron has the same nucleotide sequence, and each oligonucleotide branch of the third oligonucleotide dendron has the same nucleotide sequence, and the nucleotide sequences of the oligonucleotide branches of each of the first, second, and third oligonucleotide dendrons are different from each other. In some embodiments, each oligonucleotide branch of the first oligonucleotide dendron has the same nucleotide sequence, each oligonucleotide branch of the second oligonucleotide dendron has the same nucleotide sequence, and each oligonucleotide branch of the third oligonucleotide dendron has the same nucleotide sequence, and the nucleotide sequences of the oligonucleotide branches of the first dendron, the second dendron, and the third dendron are the same. In some embodiments, each oligonucleotide branch of the first oligonucleotide dendron has the same nucleotide sequence, each oligonucleotide branch of the second oligonucleotide dendron has the same nucleotide sequence, and each oligonucleotide branch of the third oligonucleotide dendron has the same nucleotide sequence, and the nucleotide sequences of the oligonucleotide branches of two of the oligonucleotide dendrons are the same, and are different from the nucleotide sequences of the oligonucleotide branches of the third dendron. In some embodiments, one or more of the first oligonucleotide dendron, the second oligonucleotide dendron, and the third oligonucleotide dendron comprise a plurality of oligonucleotide branches in which at least two of the oligonucleotide branches have different nucleotide sequences.

In some embodiments, a template core comprises four oligonucleotides that are hybridized to each other such that the template core comprises a first overhang, a second overhang, a third overhang, and a fourth overhang, wherein the first overhang and the second overhang are oppositely disposed, and the third overhang and the fourth overhang are oppositely disposed. The template core is then associated with a first, a second, a third, and a fourth oligonucleotide dendron, each oligonucleotide dendron comprising an oligonucleotide stem linked to a plurality of oligonucleotide branches. The oligonucleotide stem of the first oligonucleotide dendron hybridizes to the first overhang of the template core, the oligonucleotide stem of the second oligonucleotide dendron hybridizes to the second overhang of the template core, the oligonucleotide stem of the third oligonucleotide dendron hybridizes to the third overhang of the template core, and the oligonucleotide stem of the fourth oligonucleotide dendron hybridizes to the fourth overhang of the template core to create an oligonucleotide dendrimer. A plurality of the oligonucleotide dendrimers is contacted with a plurality of first SNAs, second SNAs, third SNAs, and fourth SNAs, each SNA comprising a nanoparticle core and a plurality of oligonucleotides attached to the external surface of the nanoparticle core, such that one or more oligonucleotide branches of a first oligonucleotide dendron hybridize to the plurality of oligonucleotides of the first SNA, one or more oligonucleotide branches of a second oligonucleotide dendron hybridize to the plurality of oligonucleotides of the second SNA, one or more oligonucleotide branches of a third oligonucleotide dendron hybridize to the plurality of oligonucleotides of the third SNA, and one or more oligonucleotide branches of a fourth oligonucleotide dendron hybridize to the plurality of oligonucleotides of the fourth SNA to create a multicomponent assembly. In some embodiments, each oligonucleotide branch of the first oligonucleotide dendron has the same nucleotide sequence, each oligonucleotide branch of the second oligonucleotide dendron has the same nucleotide sequence, each oligonucleotide branch of the third oligonucleotide dendron has the same nucleotide sequence, and each oligonucleotide branch of the fourth oligonucleotide dendron has the same nucleotide sequence, and the nucleotide sequences of the oligonucleotide branches of each of the first, second, third, and fourth oligonucleotide dendrons are different from each other. In some embodiments, each oligonucleotide branch of the first oligonucleotide dendron has the same nucleotide sequence, each oligonucleotide branch of the second oligonucleotide dendron has the same nucleotide sequence, each oligonucleotide branch of the third oligonucleotide dendron has the same nucleotide sequence, and each oligonucleotide branch of the third oligonucleotide dendron has the same nucleotide sequence, and the nucleotide sequences of the oligonucleotide branches of the first dendron, the second dendron, the third dendron, and the fourth dendron are the same. In some embodiments, each oligonucleotide branch of the first oligonucleotide dendron has the same nucleotide sequence, each oligonucleotide branch of the second oligonucleotide dendron has the same nucleotide sequence, each oligonucleotide branch of the third oligonucleotide dendron has the same nucleotide sequence, and each oligonucleotide branch of the fourth oligonucleotide dendron has the same nucleotide sequence, and the nucleotide sequences of the oligonucleotide branches of two of the oligonucleotide dendrons are the same, and are different from the nucleotide sequences of the oligonucleotide branches of the other two oligonucleotide dendrons. Thus, in various embodiments, the nucleotide sequences of the oligonucleotide stems of three of the oligonucleotide dendrons are the same while the nucleotide sequences of the oligonucleotide stems of the fourth oligonucleotide dendron is different; or the nucleotide sequences of the oligonucleotide stems of a first pair of oligonucleotide dendrons are the same and the nucleotide sequences of the oligonucleotide stems of a second pair of oligonucleotide dendrons are the same, but the nucleotide sequences of the first pair and the second pair are different; or the nucleotide sequences of the oligonucleotide stems of a first pair of oligonucleotide dendrons are the same while the nucleotide sequences of the oligonucleotide stems of a third oligonucleotide dendron and a fourth oligonucleotide dendron are different; or the nucleotide sequences of all four oligonucleotide dendrons are different from each other. In various embodiments, one or more of the first oligonucleotide dendron, the second oligonucleotide dendron, the third oligonucleotide dendron, and the fourth oligonucleotide dendron comprise a plurality of oligonucleotide branches in which at least two of the oligonucleotide branches have different nucleotide sequences.

In further embodiments the disclosure contemplates the use of template cores comprising 5, 6, or 7 overhangs, to which a variety of oligonucleotide dendrons as described herein are attached. Such resulting oligonucleotide dendrimers can then be combined with a plurality of SNAs as described herein to create multicomponent assemblies. The disclosure therefore contemplates template cores comprising 2, 3, 4, 5, 6, 7, or more overhangs in a target orientation, wherein an oligonucleotide dendron, each of which comprises an oligonucleotide stem linked to a plurality of oligonucleotide branches as described herein, is attached to each overhang. In various embodiments, each of the plurality of oligonucleotide branches of each oligonucleotide dendron have identical nucleotide sequences. In further embodiments, it is contemplated that with respect to pluralities of oligonucleotide branches of an oligonucleotide dendrimer (for example, when an oligonucleotide dendrimer comprises two oligonucleotide dendrons, three oligonucleotide dendrons, four oligonucleotide dendrons, five oligonucleotide dendrons, six oligonucleotide dendrons, seven oligonucleotide dendrons, or more), at least one of the pluralities of oligonucleotide branches has nucleotide sequences that are different than nucleotides sequences of at least one other plurality of oligonucleotide branches in the oligonucleotide dendrimer.

Structures of the disclosure are generally formed via hybridization of oligonucleotides. It is understood in the art that the sequence of an oligonucleotide need not be 100% complementary to that of another oligonucleotide to be specifically hybridizable. Moreover, oligonucleotides may hybridize over one or more segments such that intervening or adjacent segments are not involved in the hybridization event (e.g., an overhang or a stem-loop structure may form). In general, hybridization results in the formation of a duplex region in two complementary or sufficiently complementary oligonucleotides that form base pairs with one another, either by Watson-Crick base pairing or any other manner that allows for a stabilized duplex between oligonucleotide strands that are complementary or sufficiently complementary. “Sufficiently complementary” refers to the degree of complementarity between two nucleotide sequences such that a stable duplex is formed under the conditions in which the duplex is used. In various embodiments, sufficiently complementary nucleotide sequences are sequences that are or are at least 50%, 55%, 60%, 65%, 70%, 75%, 80%, 85%, 90%, 95%, or 99% complementary within a duplex. In some embodiments, sufficiently complementary nucleotide sequences are sequences that are 100% complementary within a duplex.

In some embodiments, oligonucleotide (e.g., DNA) dendrimers are synthesized by hybridizing multi-generation oligonucleotide (e.g., DNA) dendrons to a multi-arm oligonucleotide (e.g., DNA) template. The hybridization between complementary sequences with more than 18 base pairs occurs efficiently in a 0.5 M sodium chloride solution at room temperature. This produces oligonucleotide (e.g., DNA) dendrimers with molecularly defined architectures that can be programmed in a modular manner by changing the structure of the individual dendrons and templates. On the ends of the dendrons and subsequent dendrimers are six-base pair sticky ends that can interact with complementary strands on another nanoscale motif. By combining solutions of these dendrimers and other oligonucleotide (e.g., DNA)-functionalized nanoparticles, one can form extended nanoparticle assemblies and crystal structures in a highly modular and programmable fashion. Using toehold-mediated strand displacement reactions, these supramolecular oligonucleotide (e.g., DNA) dendrimers can be reversibly (dis) assembled, allowing the overall colloidal superlattice architecture to be reconfigured over multiple cycles. Individual dendrons that comprise the oligonucleotide (e.g., DNA) dendrimers can be designed to have orthogonal sticky ends enabling the ability to program multicomponent and multifunctional assemblies.

### Spherical Nucleic Acids (SNAs)

In any of the aspects or embodiments of the disclosure, a spherical nucleic acid (SNA) comprises a nanoparticle core and one or a plurality of oligonucleotides attached to the external surface of the nanoparticle core. In any of the aspects or embodiments of the disclosure, one or more oligonucleotides associated with a SNA are single stranded and hybridize with an oligonucleotide dendrimer as described herein.

SNAs with dendritic ligands are also contemplated for use according to the disclosure, and are described in U.S. Patent Application Publication No. 2021/0122778, which is incorporated herein by reference in its entirety.

SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of SNAs, each SNA comprising one or a plurality of oligonucleotides attached thereto. Thus, in some embodiments, the size of the plurality of SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the one or plurality of oligonucleotides attached thereto.

### Oligonucleotides

The disclosure provides spherical nucleic acids (SNAs) comprising a nanoparticle core and one or a plurality of oligonucleotides attached to the external surface of the nanoparticle core and extending outward from the nanoparticle core. Oligonucleotide dendrons also comprise oligonucleotides, and are nucleic acid structures comprising an oligonucleotide stem to which a plurality of oligonucleotide branches is linked. It will be understood that all features of oligonucleotides described herein (e.g., type (DNA/RNA), single/double stranded, length, sequence, modified forms) apply to all oligonucleotides described herein, including oligonucleotide dendrimers, oligonucleotide dendrons, oligonucleotide stems, overhangs, and oligonucleotide branches. An “oligonucleotide dendrimer” as used herein is a structure comprising at least two oligonucleotide dendrons (which may be the same or different) that are associated with each other (e.g., through hybridization) through a template core.

In various embodiments, an oligonucleotide dendron comprises about 2 to about 27 branches. In further embodiments, an oligonucleotide dendron comprises about 2 to about 25, or about 2 to about 23, or about 2 to about 20, or about 2 to about 18, or about 2 to about 16, or about 2 to about 15, or about 2 to about 13, or about 2 to about 10, or about 2 to about 8, or about 2 to about 7, or about 2 to about 5, or about 2 to about 4, or about 2 to about 3 oligonucleotide branches. In further embodiments, an oligonucleotide dendron comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or at least 27 oligonucleotide branches. In further embodiments, an oligonucleotide dendron comprises less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3 oligonucleotide branches. In some embodiments, an oligonucleotide dendron comprises or consists of 2, 3, 4, 5, 6, 7, 8, 9, 10, 11, 12, 13, 14, 15, 16, 17, 18, 19, 20, 21, 22, 23, 24, 25, 26, or 27 oligonucleotide branches. In further embodiments, an oligonucleotide dendron comprises 2, 3, 4, 6, 8, 9, 12, 18, or 27 oligonucleotide branches. In still further embodiments, an oligonucleotide dendron consists of 2, 3, 4, 6, 8, 9, 12, 18, or 27 oligonucleotide branches. In some embodiments, an oligonucleotide dendron consists of 6 branches. In some embodiments, an oligonucleotide dendron consists of 9 branches.

Oligonucleotides (e.g., an oligonucleotide attached to the external surface of a nanoparticle core, an oligonucleotide dendrimer, an oligonucleotide dendron, an oligonucleotide stem, an oligonucleotide branch, an overhang, or an oligonucleotide that is not a dendron) contemplated for use according to the disclosure include, in various embodiments, DNA oligonucleotides, RNA oligonucleotides, modified forms thereof, or a combination thereof. Thus, in some embodiments, the oligonucleotide dendron is a DNA dendron, a RNA dendron, a modified oligonucleotide dendron, or a combination thereof. In some embodiments, the oligonucleotide stem is RNA and each oligonucleotide branch that is attached to the oligonucleotide stem is DNA. In some embodiments, the oligonucleotide stem is DNA and each oligonucleotide branch that is attached to the oligonucleotide stem is RNA. Thus, in any of the aspects or embodiments of the disclosure, the oligonucleotide stem portion of an oligonucleotide dendron may be a different nucleic acid class than the oligonucleotide branches that are attached to the oligonucleotide stem, but each oligonucleotide branch in the oligonucleotide dendron is the same nucleic acid class (e.g., the oligonucleotide stem can be DNA while each oligonucleotide branch is RNA).

In any aspects or embodiments described herein, an oligonucleotide is single-stranded, double-stranded, or partially double-stranded. Thus, in various embodiments, oligonucleotide stems and oligonucleotide branches can be single, double, or partially double stranded. In some embodiments, the oligonucleotide stem is used to hybridize the oligonucleotide dendron to a template core structure to form an oligonucleotide dendrimer, while the oligonucleotide branches remain unhybridized. Similarly, in any of the aspects or embodiments of the disclosure, the oligonucleotide branches are used to hybridize to complementary structures (e.g., an oligonucleotide that is attached to a nanoparticle core of a SNA). Modified forms of oligonucleotides are also contemplated which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁-6-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂),CH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzox-azin-2 (3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 10 nucleotides to about 100 nucleotides in length. More specifically, an oligonucleotide of the disclosure is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. In further embodiments, an oligonucleotide of the disclosure is about 5 nucleotides to about 1000 nucleotides in length. In further embodiments, an oligonucleotide of the disclosure is about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length.

In various embodiments, an oligonucleotide stem of the disclosure is about 1-50 nucleotides, about 1-40 nucleotides, about 1-30 nucleotides, about 1-20 nucleotides, about 1-10 nucleotides, about 5-50 nucleotides, about 5-40 nucleotides, about 5-35 nucleotides, about 5-30 nucleotides, about 5-25 nucleotides, about 5-20 nucleotides, about 5-10 nucleotides, about 10-15 nucleotides, about 10-20 nucleotides, about 10-25 nucleotides, or about 10-30 nucleotides in length. In some embodiments, an oligonucleotide stem of the disclosure is or is about 15 nucleotides in length. In some embodiments, an oligonucleotide stem of the disclosure is or is about 18 nucleotides in length. In further embodiments, an oligonucleotide branch of the disclosure is about 1-30 nucleotides, about 1-25, about 1-20 nucleotides, about 1-15 nucleotides, about 1-10 nucleotides, about 1-5 nucleotides, about 5-10 nucleotides, about 5-15 nucleotides, about 5-20 nucleotides, about 5-25 nucleotides, about 5-30 nucleotides, about 10-15 nucleotides, about 10-20 nucleotides, about 10-25 nucleotides, or about 10-30 nucleotides in length. In some embodiments, an oligonucleotide branch of the disclosure is or is about 10 nucleotides in length.

The disclosure also provides template core structures comprising at least two oligonucleotides that hybridize to each other and generate two or more overhangs in a target orientation. For example and without limitation, and as described herein, the disclosure contemplates template core structures comprising or consisting of 2, 3, 4, 5, 6, 7 or more oligonucleotides that hybridize to each other and create 2, 3, 4, 5, 6, 7, or more overhangs, respectively. When a template core comprises or consists of two oligonucleotides that are hybridized to each other (i.e., a two-way junction), then in some embodiments the target orientation is one in which the first overhang and the second overhang are oppositely disposed. When a template core comprises or consists of three oligonucleotides that are hybridized to each other (i.e., a three-way junction), then in some embodiments the target orientation is one in which the first overhang, the second overhang, and the third overhang are in a radial (e.g., a Y-shaped) orientation. When a template core comprises or consists of four oligonucleotides that are hybridized to each other (i.e., a four-way junction), then in some embodiments the target orientation is one in which the first overhang and the second overhang are oppositely disposed, and the third overhang and the fourth overhang are oppositely disposed. In various embodiments, the length of each overhang is or is about 4-40 nucleotides, 4-30 nucleotides, 4-20 nucleotides, 4-10 nucleotides, 5-40 nucleotides, 5-30 nucleotides, 5-20 nucleotides, 10-40 nucleotides, 10-30 nucleotides, 10-20 nucleotides, 15-40 nucleotides, 15-30 nucleotides, or 15-20 nucleotides in length. In further embodiments, each overhang is or is at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, each overhang is 18 nucleotides in length.

Nanoparticle surface density. A surface density adequate to make the nanoparticles stable and the conditions necessary to obtain it for a desired combination of nanoparticles and oligonucleotides can be determined empirically. In some embodiments, one oligonucleotide is attached to the external surface of a nanoparticle core. In further embodiments, about 2 to about 200 oligonucleotides are attached to the external surface of a nanoparticle core. In further embodiments, about 2 to about 190, or about 2 to about 180, or about 2 to about 170, or about 2 to about 160, or about 2 to about 150, or about 2 to about 140, or about 2 to about 130, or about 2 to about 120, or about 2 to about 110, or about 2 to about 100, or about 2 to about 90, or about 2 to about 80, or about 2 to about 70, or about 2 to about 60, or about 2 to about 50, or about 2 to about 40, or about 2 to about 30, or about 2 to about 20, or about 2 to about 10, or about 10 to about 100, or about 10 to about 90, or about 10 to about 80, or about 10 to about 70, or about 10 to about 60, or about 10 to about 50, or about 10 to about 40, or about 10 to about 30, or about 10 to about 20, or about 20 to about 100, or about 20 to about 90, or about 20 to about 80, or about 20 to about 70, or about 20 to about 60, or about 20 to about 50, or about 20 to about 40, or about 20 to about 30 oligonucleotides are attached to the external surface of a nanoparticle core.

In further embodiments, oligonucleotides are attached to the nanoparticle core at a surface density of at least about 2 pmoles/cm². In some aspects, the surface density is at least 15 pmoles/cm². Methods are also provided wherein the oligonucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. Alternatively, the density of oligonucleotides attached to the SNA is measured by the number of oligonucleotides attached to the SNA. With respect to the surface density of oligonucleotides attached to an SNA, it is contemplated that a SNA as described herein comprises about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In further embodiments, a SNA consists of 1, 2, 3, 4, 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20 or more oligonucleotides. In some embodiments, the shell of oligonucleotides attached to the nanoparticle core of the SNA comprises at least 20 oligonucleotides. In some embodiments, the shell of oligonucleotides attached to the nanoparticle core of the SNA consists of 1, 2, 3, 4, 5, 6, 7, 8, 9, 10, 11, 12, 13, 14, 15, 16, 17, 18, 19, or 20 oligonucleotides.

Spacers. In some aspects, an oligonucleotide is attached to a nanoparticle through a spacer. “Spacer” as used herein means a moiety that serves to increase distance between the nanoparticle and the oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo (ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotide to perform an intended function (e.g., inhibit gene expression). In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 20 nucleotides, 10-30 nucleotides, or even greater than 30 nucleotides.

Oligonucleotide attachment to a nanoparticle core. Oligonucleotides contemplated for use according to the disclosure include those attached to a nanoparticle core through any means (e.g., covalent or non-covalent attachment). Regardless of the means by which the oligonucleotide is attached to the nanoparticle, attachment in various aspects is effected through a 5′ linkage, a 3′ linkage, some type of internal linkage, or any combination of these attachments. In some embodiments, the oligonucleotide is covalently attached to a nanoparticle. In further embodiments, the oligonucleotide is non-covalently attached to a nanoparticle.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating oligonucleotides with a liposomal particle are described in PCT/US2014/068429, which is incorporated by reference herein in its entirety.

Methods of attaching oligonucleotides to a protein core are described, e.g., in U.S. Patent Application Publication No. 2017/0232109, U.S. Patent Application Publication No. 2021/0122778, and Brodin et al., J Am Chem Soc. 137 (47): 14838-41 (2015), each of which is incorporated by reference herein in its entirety. In general, a polynucleotide can be modified at a terminus with an alkyne moiety, e.g., a DBCO-type moiety for reaction with the azide of the protein surface:

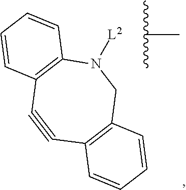

where L is a linker to a terminus of the polynucleotide. L² can be C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

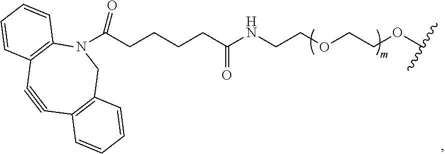

where the terminal “O” is from a terminal nucleotide on the polynucleotide. Use of this DBCO-type moiety results in a structure between the polynucleotide and the protein, in cases where a surface amine is modified, of:

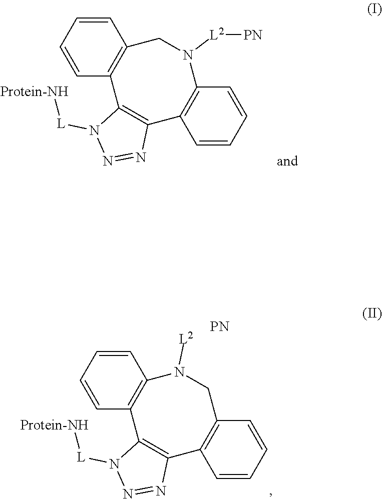

where L and L² are each independently selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and PN is the polynucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N³, X is from a surface amino group (e.g., —NH—), carboxylic group (e.g., —C(O)— or —C(O)O—), or thiol group (e.g., —S—) on the protein; L is selected from C₁₋₁₀ alkylene, —Y—C(O)—C₁₋₁₀ alkylene-Y—, and —Y—C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface moiety of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The polynucleotide can be modified to include an alkyne functional group at a terminus of the polynucleotide: Polynucleotide-L₂-X—≡—R; L² is selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the polynucleotide has a structure

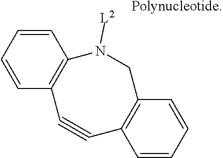

The protein, with the surface modified azide, and the polynucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl) phosphine, hydroquinone, and mixtures thereof.

The surface functional group of the protein can be attached to the polynucleotide using other attachment chemistries. For example, a surface amine can be directed conjugated to a carboxylate or activated ester at a terminus of the polynucleotide, to form an amide bond. A surface carboxylate can be conjugated to an amine on a terminus of the polynucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate.

### Nanoparticle Core

In general, nanoparticles contemplated by the disclosure include any compound or substance with a loading capacity for an oligonucleotide as described herein, including for example and without limitation, a metal, a semiconductor, a protein, a liposomal particle, a polymer-based particle (e.g., a poly (lactic-co-glycolic acid) (PLGA) particle), or insulator particle compositions. Thus, in various embodiments, the nanoparticle core is organic (e.g., a liposome), inorganic (e.g., gold, silver, or platinum), porous (e.g., silica-based or metal organic-framework-based), or hollow.

Thus, the disclosure contemplates nanoparticle cores that comprise a variety of inorganic materials including, but not limited to, metals, semi-conductor materials or ceramics as described in U.S. Patent Publication No 20030147966. For example, metal-based nanoparticles include those described herein. In various embodiments, the nanoparticle core is a metallic core, a semiconductor core, an insulator core, an upconverting core, a liposomal core, a polymer core, a metal-organic framework core, a protein core, or a combination thereof. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles. In some embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), chitosan, or a related structure. In some embodiments, the polymer is poly(lactic-co-glycolic acid) (PLGA).

Liposomal particles, for example as disclosed in International Patent Application No. PCT/US2014/068429 and U.S. Pat. No. 10,792,251 (each of which is incorporated by reference herein in its entirety) are also contemplated by the disclosure. Hollow particles, for example as described in U.S. Patent Publication Number 2012/0282186 (incorporated by reference herein in its entirety) are also contemplated herein. Liposomes of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. In various embodiments, the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, or a combination thereof.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992). In some embodiments, the nanoparticle is an iron oxide nanoparticle. In further embodiments, the nanoparticle core is gold, silver, platinum, aluminum, palladium, copper, cobalt, indium, cadmium selenide, iron oxide, fullerene, metal-organic framework, zinc sulfide, or nickel.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53:137-143 and U.S. Pat. No. 4,489,055. Methods for making nanoparticles comprising poly(D-glucaramidoamine) s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Proteins that may be used as a protein core and methods of making protein core SNAs are described, for example, in U.S. Patent Application Publication No. 2021/0122778, which is incorporated herein by reference in its entirety. Thus, in some embodiments, the nanoparticle core is a protein. In further embodiments, the nanoparticle core is a peptide core. As used herein, protein is used interchangeably with “polypeptide” and refers to one or more polymers of amino acid residues. In various embodiments of the disclosure, a protein core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. Protein/oligonucleotide core-shell nanoparticles are also generally described in U.S. Patent Application Publication No. 2017/0232109, which is incorporated by reference herein in its entirety.

Proteins are understood in the art and include without limitation an enzyme, a therapeutic protein (e.g., adenosine deaminase, phosphatase and tensin homolog (PTEN), or interleukin-2 (IL-2)), a structural protein (e.g., actin), an antibody, a storage protein (e.g., ovalbumin), a transport protein (e.g., hemoglobin), a hormone (e.g., insulin), a receptor protein (e.g., G-Protein Coupled Receptors), a motor protein (e.g., kinesin, dynein, or myosin), an immunogenic protein (e.g., ovalbumin or a stimulator of interferon genes (STING) protein) or a fluorescent protein (e.g., green fluorescent protein (GFP), mutant neon green (mNeonGreen), or ruby red protein). In various embodiments, proteins contemplated by the disclosure include without limitation those having catalytic, signaling, therapeutic, or transport activity.

Proteins of the present disclosure may be either naturally occurring or non-naturally occurring. Proteins optionally include a spacer as described herein.

Naturally occurring proteins include without limitation biologically active proteins (including antibodies) that exist in nature or can be produced in a form that is found in nature by, for example, chemical synthesis or recombinant expression techniques. Thus, a protein core of the disclosure is or comprises, in some embodiments, an antibody. Naturally occurring proteins also include lipoproteins and post-translationally modified proteins, such as, for example and without limitation, glycosylated proteins. Antibodies contemplated for use in the methods and compositions of the present disclosure include without limitation antibodies that recognize and associate with a target molecule either in vivo or in vitro.

Structural proteins contemplated by the disclosure include without limitation actin, tubulin, collagen, and elastin.

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “peptide” typically refers to short (e.g., about 2-50 amino acids in length) polypeptides/proteins. Non-naturally occurring proteins are prepared, for example, using an automated protein synthesizer or, alternatively, using recombinant expression techniques using a modified polynucleotide which encodes the desired protein.

As used herein a “fragment” of a protein is meant to refer to any portion of a protein smaller than the full-length protein or protein expression product. As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it. As used herein a “variant” refers to a protein or analog thereof that is modified to comprise additional chemical moieties not normally a part of the molecule. Such moieties may modulate, for example and without limitation, the molecule s solubility, absorption, and/or biological half-life. Moieties capable of mediating such effects are disclosed in Remington's Pharmaceutical Sciences (1980). Procedures for coupling such moieties to a molecule are well known in the art. In various aspects, proteins are modified by glycosylation, pegylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

Proteins include antibodies along with fragments and derivatives thereof, including but not limited to Fab′ fragments, F(ab)2 fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

Suitable nanoparticles are also commercially available from, for example, Ted Pella, Inc. (gold), Amersham Corporation (gold) and Nanoprobes, Inc. (gold).

As used in this specification and the appended claims, the singular forms “a,” “an,” and “the” include plural reference unless the context clearly dictates otherwise.

The terms “polynucleotide” and “oligonucleotide” are interchangeable as used herein.

The term “about” as used herein in reference to a value, encompasses from 90% to 110% of that value (e.g., a nanoparticle that is about 100 nanometers (nm) in diameter refers to a nanoparticle that is 90 nm to 110 nm in diameter).

### EXAMPLES

### Example 1

### Dendron Synthesis

DNA dendrons were synthesized using a previously established synthesis [Distler, M. E.; Teplensky, M. H.; Bujold, K. E.; Kusmierz, C. D.; Evangelopoulos, M.; Mirkin, C. A., DNA Dendrons as Agents for Intracellular Delivery. Journal of the American Chemical Society 2021, 143 (34), 13513-13518] with an automated ABI DNA synthesizer on 2000 angstrom controlled pore glass (CPG) beads, commonly used in solid-phase DNA synthesis. Phosphoramidite branching units were purchased from Glen Research and applied to this synthesis to access a dendritic DNA architecture. Optimal synthetic conditions were determined by systematically changing DNA length, sequence, flexibility, and branching unit design. The resultant molecules are purified by 8-15% denaturing polyacrylamide gel electrophoresis (PAGE). Purified products are characterized by Time of Flight Matrix-Assisted Laser Desorption/Ionization Mass Spectrometry (MALDI-TOF) (B) and PAGE (C).

It was hypothesized that DNA dendrons have the potential to program unique, orthogonal interactions in colloidal crystals, a capability that has long been elusive in this field. Individual DNA dendrons can be designed to contain orthogonal sticky ends (branches) that hybridize with different strands on DNA-functionalized NPs (FIG. 1, top). The stems of each of these dendrons are designed to form DNA junctions with one another (FIG. 1, middle). As a result, EEs and PAEs with organized clusters of orthogonal sticky ends can then direct the assembly of multiple NP components into a single, ordered, crystalline assembly (FIG. 1, bottom).

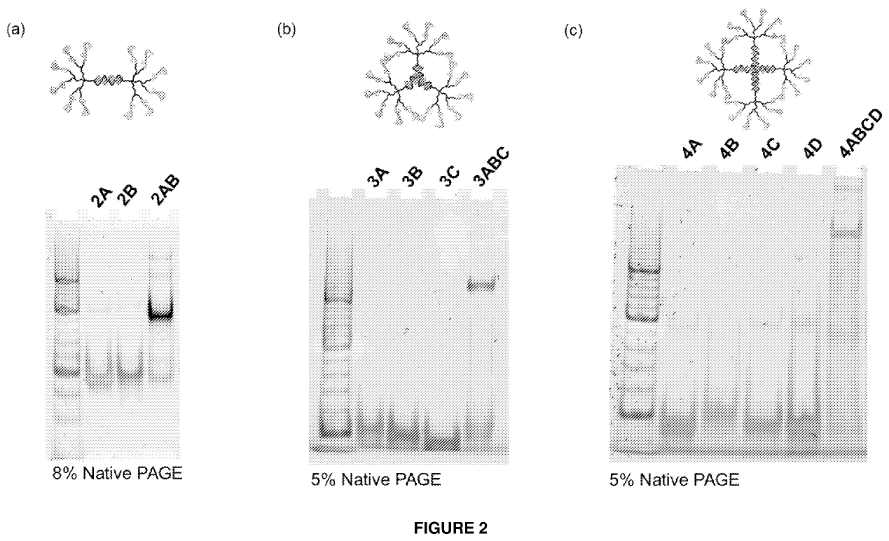

To form junctions, the DNA dendrons were annealed in 0.5 M NaCl at 95° C. for 10 minutes and cooled from 95-20° C. over 1 hour. Resultant junctions were characterized by native PAGE as shown for the 2-way junction (FIG. 2a), the 3-way junction (FIG. 2b), and the 4-way junction (FIG. 2c).

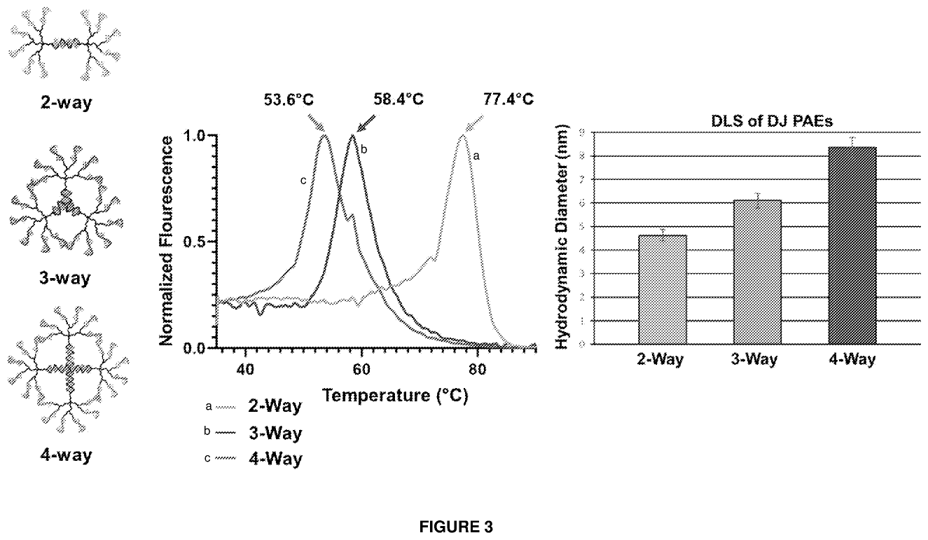

FIG. 3(a) shows DNA junction melting temperatures were determined by measuring the fluorescence signal of an intercalating dye, SYBR Green. As the junctions were heated and de-hybridized, the fluorescence signal decreases. The derivative of that melting curve indicates the two-way junction has a Tₘ of 77.4° C., the three-way junction has a Tₘ of 58.4° C., and the four-way junction has a Tₘ of 53.6° C. (FIG. 3b) The size of each junction was determined by dynamic light scattering (DLS). The junctions have hydrodynamic diameters of 4.3 nm, 6.1 nm, and 8.2 nm, for the two-, three-, and four-way junctions, respectively.

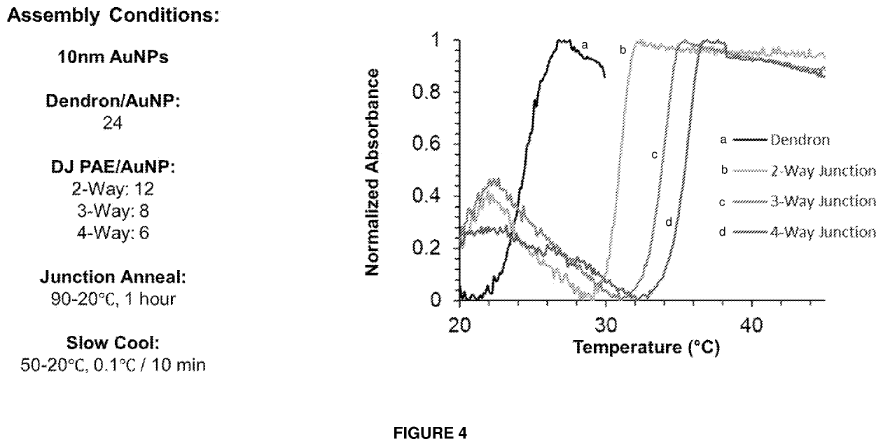

DNA functionalized 10 nm AuNPs were assembled with each of the DNA junctions (2-, 3-, 4-way) in 0.5M NaCl, 10×PBS, and 0.1% SDS. The total number of sticky ends were held constant which is key to compare the thermostabilities of the resultant assemblies. The melting temperature of each assembly was determined by a UV-vis melt experiment. A characteristic increase in the melting temperature was observed as the valency and size of the DNA junction PAEs (DJ PAEs) increased. See FIG. 4.

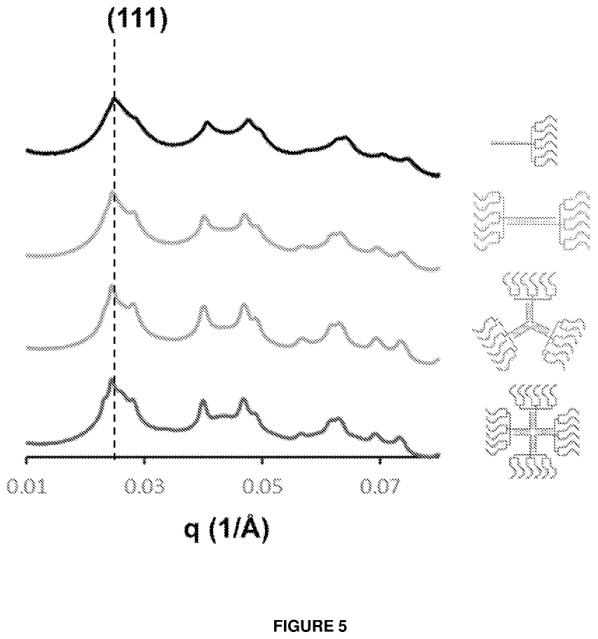

FIG. 5 shows that DNA functionalized 10 nm AuNPs were assembled with each of the DNA junctions. While the melting temperature changes (see FIG. 4), the crystal structure remains constant due to the vast size difference between the Au PAEs and the DJ PAEs, forming an FCC crystal.

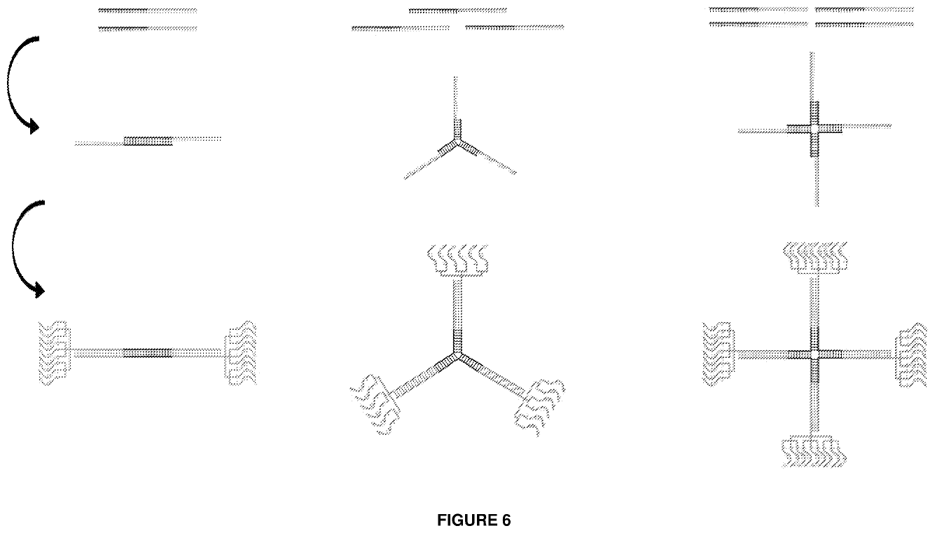

A new DNA dendron design that allows greater programmability over the DJ PAE architecture is shown in FIG. 6. Linear DNA strands were used to form a template core in the form of a DNA junction. The templates have 18 base overhangs to which the stems of the dendrons can hybridize. This approach provided more design handles that allows us to more easily tune the thermostabilities and organizations of resultant assemblies. To form the DJ PAE, the DNA dendrons were annealed in 0.5 M NaCl at 95° C. for 10 min and cooled from 95-20° C. over 1 hr.

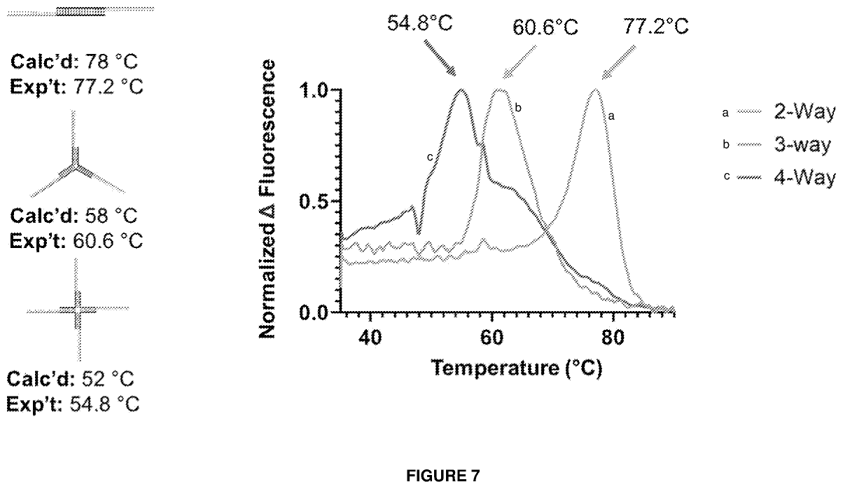

Melt data of the DNA junction templates were collected through previously described methods (see FIG. 3). An expected decrease in melting temperature was observed as the junction valency was increased (FIG. 7).

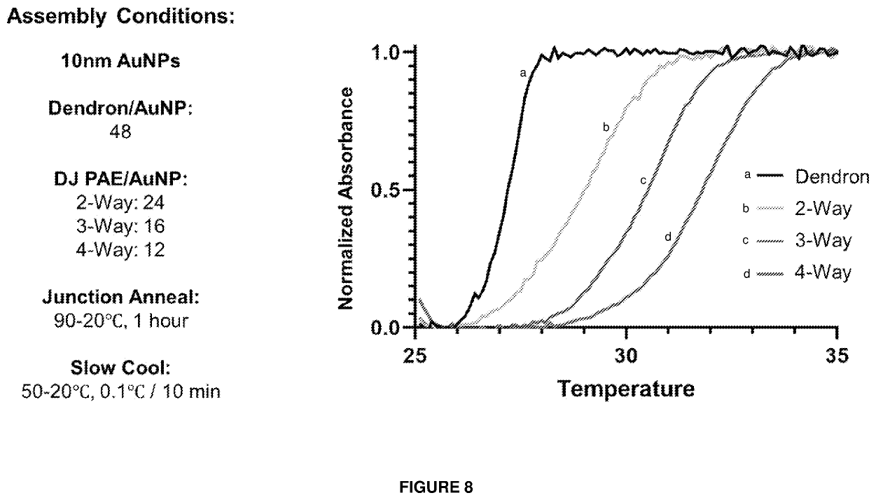

Larger DJ PAEs were assembled with 10 nm AuNP PAEs to form colloidal crystals (FIG. 8). Melting experiments revealed an expected increase in colloidal crystal thermostability as the junction core increased in valency. This was expected because the 4-way DJ PAE would have significantly more sticky ends per bonding moiety, thereby forming more favorable interactions with the AuNP PAEs.

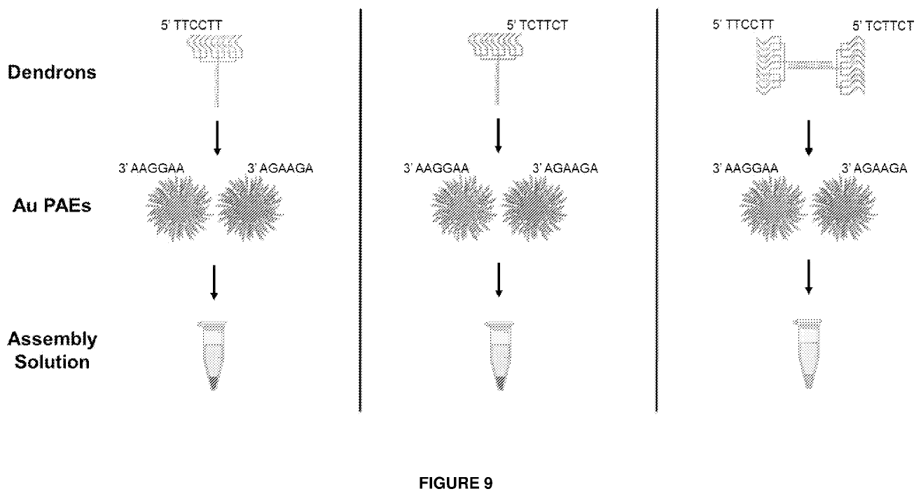

FIG. 9 shows an experimental design for determining if two types of nanoparticles can be incorporated into a single colloidal crystal using the DJ PAE. Both NP types were present in solution. (FIG. 9, Left) Only one dendron was present so only one particle type can assemble. (FIG. 9, Middle) Only the other dendron was present so the other NP type could assemble. (FIG. 9, Right) Both dendrons were present and formed a DNA junction so that both particles can be assembled into a single colloidal crystal.

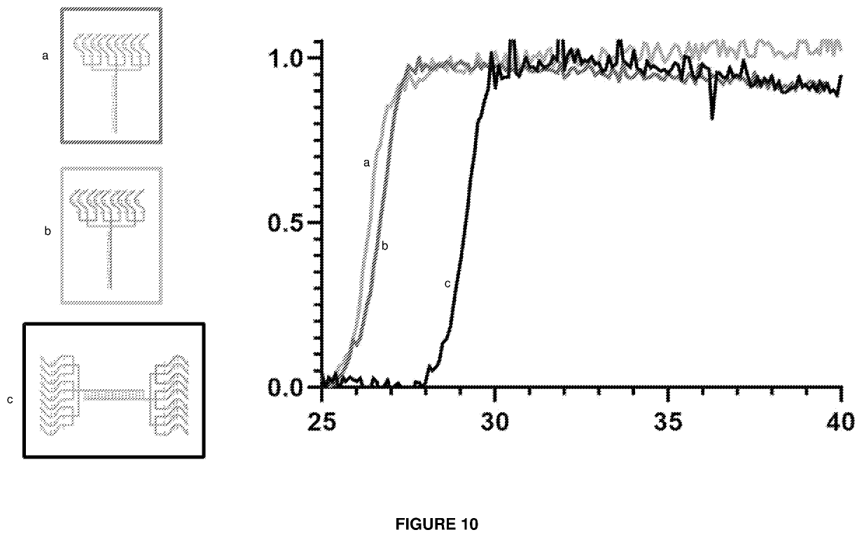

FIG. 10 shows results of melting experiments that were conducted on the assemblies from FIG. 9. In the samples that only contained a single dendron, the melting temperature was around 26 C. These results matched previous data for single dendrons used in NP assembly. However, when both dendrons were present and formed a DNA junction, s single, significantly higher melting transition was observed, indicating that both particles were incorporated into a single, more stable, crystal structure. This was the first proof of concept to show that by using DJ PAEs a completely new assembly is able to be synthesized, which can contain two different components

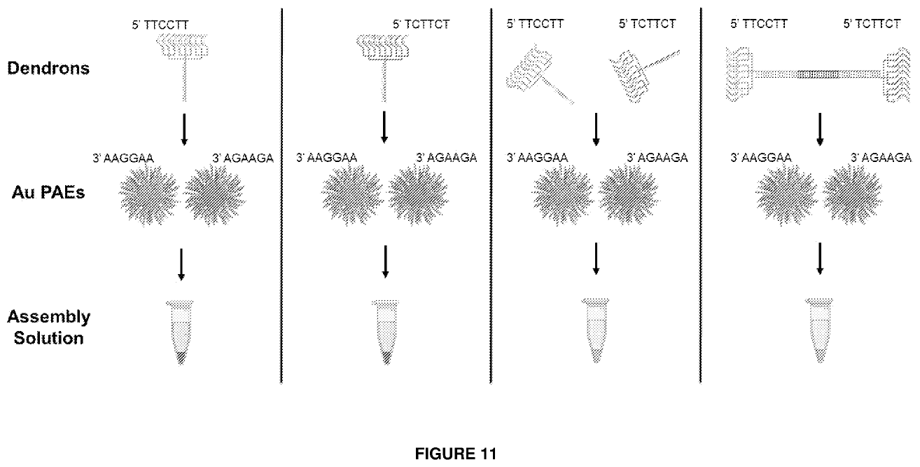

FIG. 11 depicts an experimental design for determining if two types of nanoparticles can be incorporated into a single colloidal crystal using the larger DJ PAE approach. Both NP types were present in solution. (FIG. 11, Left) Only one dendron was present so only one particle type can assemble. (FIG. 11, Middle-Left)) Only the other dendron was present so the other NP type could assemble. (FIG. 11, Middle-Right) Both dendrons were present but no DNA junction template was provided. This means that the dendrons would not be able to form into a DJ PAE and therefore should behave separately in solution. (FIG. 11, Right) Both dendrons and the DNA junction template were present. Therefore, the entire DJ PAE formed and both particles are incorporated into a single crystal.

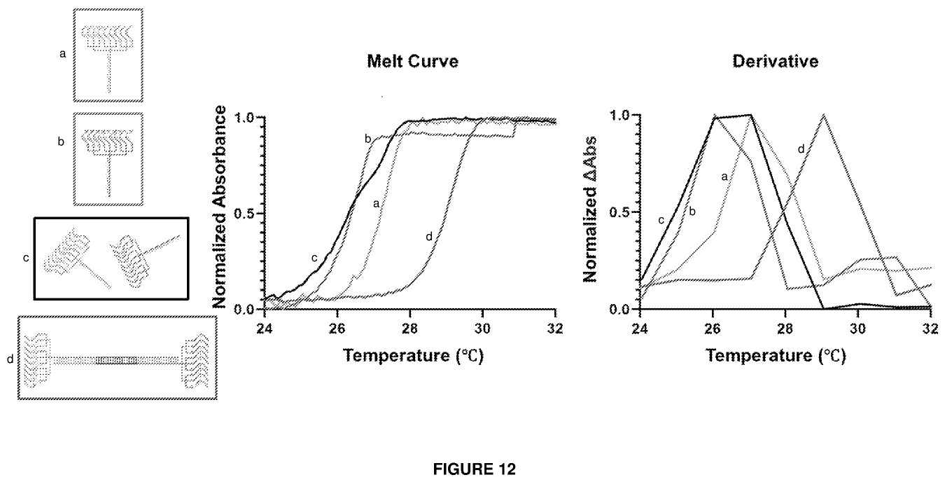

FIG. 12 shows results of melting experiments that were conducted on the assemblies from FIG. 11. Single dendron containing assemblies had low melting temperatures that match previous results. Interestingly, when both dendrons were present but had no DNA junction core to connect them (black trace), two separate melting transitions formed: one that matched the melting temperature of the orange trace, and one that matched the melting temperature of the blue trace. This indicated that without the DNA junction core, two separate colloidal crystals form with their own distinct thermostabilities. Finally, when all components are combined to form the complete DJ PAE, a significantly higher, single melting transition was observed. This data supported that only when all components are present do the colloidal crystals contain both nanoparticle types within a single colloidal crystal.

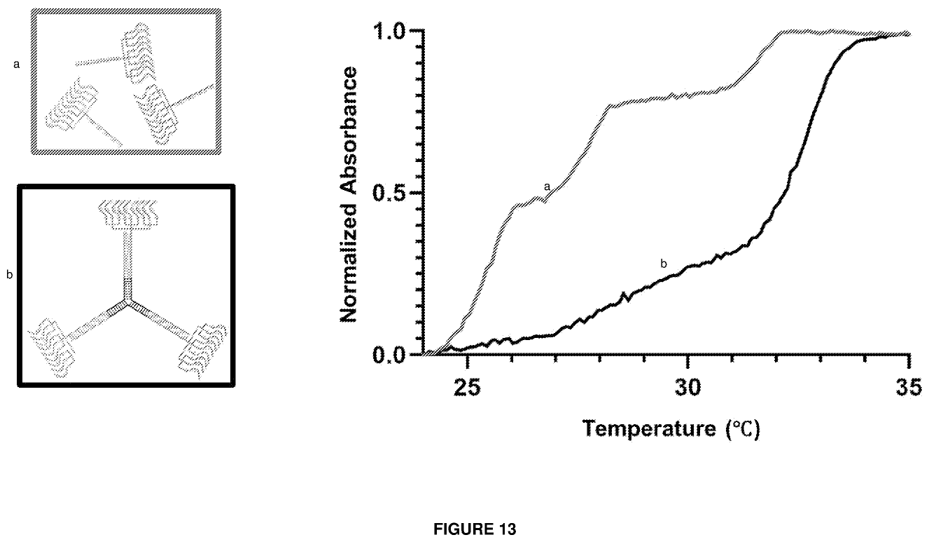

A similar melting experiment was conducted on assemblies that were formed containing three unique AuNP PAEs (FIG. 13). The assembly that contained all three dendrons, but no junction core experienced three separate melting transitions. This indicated that while assemblies formed under these conditions, each unique particle type formed a separate colloidal crystal (red trace). When all three dendrons and the junction core were present in the assembly solution, the fully composed DJ PAE formed. As a result, a single melting transition was observed, indicating that all three particle types were incorporated into a single colloidal crystal (black trace).

What is claimed is:

1. An oligonucleotide dendrimer comprising:
a template core comprising a first oligonucleotide and a second oligonucleotide that are hybridized to each other such that the template core comprises a first overhang and a second overhang, wherein the first overhang and the second overhang are arranged in a target orientation;
a first oligonucleotide dendron comprising an oligonucleotide stem linked to a plurality of oligonucleotide branches, wherein the oligonucleotide stem is hybridized to the first overhang; and
a second oligonucleotide dendron comprising an oligonucleotide stem linked to a plurality of oligonucleotide branches, wherein the oligonucleotide stem is hybridized to the second overhang.

2. The oligonucleotide dendrimer of claim 1, wherein the plurality of oligonucleotide branches of the first oligonucleotide dendron and the plurality of oligonucleotide branches of the second oligonucleotide dendron have identical nucleotide sequences.

3. The oligonucleotide dendrimer of claim 1, wherein the plurality of oligonucleotide branches of the first oligonucleotide dendron and the plurality of oligonucleotide branches of the second oligonucleotide dendron have different nucleotide sequences.

4. The oligonucleotide dendrimer of any one of claims 1-3, wherein in the target orientation the first overhang and the second overhang are oppositely disposed.

5. The oligonucleotide dendrimer of any one of claims 1-4, wherein the template core comprises a third oligonucleotide that is hybridized to the first oligonucleotide and the second oligonucleotide such that the template core comprises a third overhang, wherein the first overhang, the second overhang, and the third overhang are arranged in a target orientation,
the oligonucleotide dendrimer further comprising a third oligonucleotide dendron comprising an oligonucleotide stem linked to a plurality of oligonucleotide branches, wherein the oligonucleotide stem is hybridized to the third overhang.

6. The oligonucleotide dendrimer of claim 5, wherein the plurality of oligonucleotide branches of the first oligonucleotide dendron, the plurality of oligonucleotide branches of the second oligonucleotide dendron, and the plurality of oligonucleotide branches of the third oligonucleotide dendron have identical nucleotide sequences.

7. The oligonucleotide dendrimer of claim 5, wherein the plurality of oligonucleotide branches of the first oligonucleotide dendron, the plurality of oligonucleotide branches of the second oligonucleotide dendron, and the plurality of oligonucleotide branches of the third oligonucleotide dendron have different nucleotide sequences.

8. The oligonucleotide dendrimer of claim 5, wherein the plurality of oligonucleotide branches of the first oligonucleotide dendron and the plurality of oligonucleotide branches of the second oligonucleotide dendron have identical nucleotide sequences, but the plurality of oligonucleotide branches of the third oligonucleotide dendron has nucleotide sequences that are different than the plurality of oligonucleotide branches of the first oligonucleotide dendron and the plurality of oligonucleotide branches of the second oligonucleotide dendron.

9. The oligonucleotide dendrimer of any one of claims 5-7, wherein in the target orientation the first overhang, the second overhang, and the third overhang are in a radial orientation.

10. The oligonucleotide dendrimer of any one of claims 5-8, wherein the template core comprises a fourth oligonucleotide and hybridization of the first oligonucleotide, the second oligonucleotide, the third oligonucleotide, and the fourth oligonucleotide creates a fourth overhang, wherein the first overhang, the second overhang, the third overhang, and the fourth overhang are arranged in a target orientation,
the oligonucleotide dendrimer further comprising a fourth oligonucleotide dendron comprising an oligonucleotide stem linked to a plurality of oligonucleotide branches, wherein the oligonucleotide stem is hybridized to the fourth overhang.

11. The oligonucleotide dendrimer of claim 10, wherein the plurality of oligonucleotide branches of the first oligonucleotide dendron, the plurality of oligonucleotide branches of the second oligonucleotide dendron, the plurality of oligonucleotide branches of the third oligonucleotide dendron, and the plurality of oligonucleotide branches of the fourth oligonucleotide dendron have identical nucleotide sequences.

12. The oligonucleotide dendrimer of claim 10, wherein the plurality of oligonucleotide branches of the first oligonucleotide dendron, the plurality of oligonucleotide branches of the second oligonucleotide dendron, the plurality of oligonucleotide branches of the third oligonucleotide dendron, and the plurality of oligonucleotide branches of the fourth oligonucleotide dendron have different nucleotide sequences.

13. The oligonucleotide dendrimer of claim 10, wherein:
the plurality of oligonucleotide branches of the first oligonucleotide dendron and the plurality of oligonucleotide branches of the second oligonucleotide dendron have identical nucleotide sequences;
the plurality of oligonucleotide branches of the third oligonucleotide dendron and the plurality of oligonucleotide branches of the fourth oligonucleotide dendron have identical nucleotide sequences, and
the plurality of oligonucleotide branches of the first oligonucleotide dendron and the plurality of oligonucleotide branches of the second oligonucleotide dendron have nucleotide sequences that are different from nucleotide sequences of the plurality of oligonucleotide branches of the first oligonucleotide dendron and the plurality of oligonucleotide branches of the second oligonucleotide dendron.

14. The oligonucleotide dendrimer of any one of claims 10-13, wherein in the target orientation the first overhang and the second overhang are oppositely disposed, and the third overhang and the fourth overhang are oppositely disposed.

15. The oligonucleotide dendrimer of any one of claims 1-14, wherein the first overhang is about 4 to about 40 nucleotides in length.

16. The oligonucleotide dendrimer of any one of claims 1-15, wherein the first overhang is about 18 nucleotides in length.

17. The oligonucleotide dendrimer of any one of claims 1-16, wherein the second overhang is about 4 to about 40 nucleotides in length.

18. The oligonucleotide dendrimer of any one of claims 1-17, wherein the second overhang is about 18 nucleotides in length.

19. The oligonucleotide dendrimer of any one of claims 5-18, wherein the third overhang is about 4 to about 40 nucleotides in length.

20. The oligonucleotide dendrimer of any one of claims 5-17, wherein the third overhang is about 18 nucleotides in length.

21. The oligonucleotide dendrimer of any one of claims 10-20, wherein the fourth overhang is about 4 to about 40 nucleotides in length.

22. The oligonucleotide dendrimer of any one of claims 10-21, wherein the fourth overhang is about 18 nucleotides in length.

23. The oligonucleotide dendrimer of any one of claims 1-22, wherein the first oligonucleotide and the second oligonucleotide comprise DNA, RNA, a modified oligonucleotide, or a combination thereof.

24. The oligonucleotide dendrimer of any one of claims 5-23, wherein the third oligonucleotide comprises DNA, RNA, a modified oligonucleotide, or a combination thereof.

25. The oligonucleotide dendrimer of any one of claims 10-24, wherein the fourth oligonucleotide comprises DNA, RNA, a modified oligonucleotide, or a combination thereof.

26. The oligonucleotide dendrimer of any one of claims 10-25, wherein the template core comprises a fifth oligonucleotide and hybridization of the first oligonucleotide, the second oligonucleotide, the third oligonucleotide, the fourth oligonucleotide, and the fifth oligonucleotide creates a fifth overhang, wherein the first overhang, the second overhang, the third overhang, the fourth overhang, and the fifth overhang are arranged in a target orientation,
the oligonucleotide dendrimer further comprising a fifth oligonucleotide dendron comprising an oligonucleotide stem linked to a plurality of oligonucleotide branches, wherein the oligonucleotide stem is hybridized to the fifth overhang.

27. The oligonucleotide dendrimer of claim 26, wherein the template core comprises a sixth oligonucleotide and hybridization of the first oligonucleotide, the second oligonucleotide, the third oligonucleotide, the fourth oligonucleotide, the fifth oligonucleotide, and the sixth oligonucleotide creates a sixth overhang, wherein the first overhang, the second overhang, the third overhang, the fourth overhang, the fifth overhang, and the sixth overhang are arranged in a target orientation,
the oligonucleotide dendrimer further comprising a sixth oligonucleotide dendron comprising an oligonucleotide stem linked to a plurality of oligonucleotide branches, wherein the oligonucleotide stem is hybridized to the sixth overhang.

28. The oligonucleotide dendrimer of claim 27, wherein the template core comprises a seventh oligonucleotide and hybridization of the first oligonucleotide, the second oligonucleotide, the third oligonucleotide, the fourth oligonucleotide, the fifth oligonucleotide, the sixth oligonucleotide, and the seventh oligonucleotide creates a seventh overhang, wherein the first overhang, the second overhang, the third overhang, the fourth overhang, the fifth overhang, the sixth overhang, and the seventh overhang are arranged in a target orientation,
the oligonucleotide dendrimer further comprising a seventh oligonucleotide dendron comprising an oligonucleotide stem linked to a plurality of oligonucleotide branches, wherein the oligonucleotide stem is hybridized to the seventh overhang.

29. A crystal comprising:
(i) a plurality of oligonucleotide dendrimers, each oligonucleotide dendrimer comprising:
a template core comprising first and second oligonucleotides hybridized to each other such that the template core has first and second overhangs, wherein the first and second overhangs are in a target orientation,
a first oligonucleotide dendron comprising an oligonucleotide stem linked to a plurality of oligonucleotide branches, with the oligonucleotide stem being hybridized to the first overhang, and
a second oligonucleotide dendron comprising an oligonucleotide stem linked to a plurality of oligonucleotide branches, with the oligonucleotide stem being hybridized to the second overhang;
(ii) a plurality of first spherical nucleic acids (SNAs), each comprising a nanoparticle core and a plurality of oligonucleotides attached to the external surface of the nanoparticle core, wherein one or more oligonucleotides of the plurality of oligonucleotides of each of the first SNAs is hybridized to one or more oligonucleotide branches of the plurality of oligonucleotide branches of at least one first oligonucleotide dendron of the plurality of oligonucleotide dendrimers; and
(iii) a plurality of second spherical nucleic acids (SNAs), each comprising a nanoparticle core and a plurality of oligonucleotides attached to the external surface of the nanoparticle core, wherein one or more oligonucleotides of the plurality of oligonucleotides of each of the second SNAs is hybridized to one or more oligonucleotide branches of the plurality of oligonucleotide branches of at least one second oligonucleotide dendron of the plurality of oligonucleotide dendrimers.

30. The crystal of claim 29, wherein in the target orientation the first overhang and the second overhang are oppositely disposed.

31. The crystal of claim 29 or claim 30, further comprising a plurality of third spherical nucleic acids (SNAs), each comprising a nanoparticle core and a plurality of oligonucleotides attached to the external surface of the nanoparticle core, wherein:
the template core of each of the plurality of oligonucleotide dendrimers comprises a third oligonucleotide that is hybridized to the first oligonucleotide and the second oligonucleotide such that the template core comprises a third overhang, wherein the first overhang, the second overhang, and the third overhang are in the target orientation,
and each of the plurality of dendrimers further comprises a third oligonucleotide dendron comprising an oligonucleotide stem linked to a plurality of oligonucleotide branches, with the oligonucleotide stem being hybridized to the third overhang, and
one or more oligonucleotides of the plurality of oligonucleotides of each of the third SNAs is hybridized to one or more oligonucleotide branches of the plurality of oligonucleotide branches of at least one third oligonucleotide dendron.

32. The crystal of claim 31, wherein in the target orientation the first overhang, the second overhang, and the third overhang are in a radial orientation.

33. The crystal of claim 31 or claim 32, further comprising a plurality of fourth spherical nucleic acids (SNAs) comprising a nanoparticle core and a plurality of oligonucleotides attached to the external surface of the nanoparticle core, wherein:
the template core of each of the plurality of oligonucleotide dendrimers comprises a fourth oligonucleotide and hybridization of the first oligonucleotide, the second oligonucleotide, the third oligonucleotide, and the fourth oligonucleotide results in a fourth overhang, wherein the first overhang, the second overhang, the third overhang, and the fourth overhang are in the target orientation,
and each of the plurality of dendrimers further comprises a fourth oligonucleotide dendron comprising an oligonucleotide stem linked to a plurality of oligonucleotide branches, with the oligonucleotide stem being hybridized to the fourth overhang, and
one or more oligonucleotides of the plurality of oligonucleotides of each of the fourth SNAs is hybridized to one or more oligonucleotide branches of the plurality of oligonucleotide branches of the fourth oligonucleotide dendron.

34. The crystal of claim 33, wherein in the target orientation the first overhang and the second overhang are oppositely disposed, and the third overhang and the fourth overhang are oppositely disposed.

35. The crystal of claim 33 or claim 34, further comprising a plurality of fifth spherical nucleic acids (SNAs) comprising a nanoparticle core and a plurality of oligonucleotides attached to the external surface of the nanoparticle core, wherein:
the template core of each of the plurality of oligonucleotide dendrimers comprises a fifth oligonucleotide and hybridization of the first oligonucleotide, the second oligonucleotide, third oligonucleotide, fourth oligonucleotide, and fifth oligonucleotide results in a fifth overhang, wherein the first overhang, the second overhang, the third overhang, the fourth overhang, and the fifth overhang are in the target orientation,
and each of the plurality of dendrimers further comprises a fifth oligonucleotide dendron comprising an oligonucleotide stem linked to a plurality of oligonucleotide branches, with the oligonucleotide stem being hybridized to the fifth overhang, and
one or more oligonucleotides of the plurality of oligonucleotides of each of the fifth SNAs is hybridized to one or more oligonucleotide branches of the plurality of oligonucleotide branches of the fifth oligonucleotide dendron.

36. The crystal of claim 35, further comprising a plurality of sixth spherical nucleic acids (SNAs) comprising a nanoparticle core and a plurality of oligonucleotides attached to the external surface of the nanoparticle core, wherein:
the template core of each of the plurality of oligonucleotide dendrimers comprises a sixth oligonucleotide and hybridization of the first oligonucleotide, the second oligonucleotide, the third oligonucleotide, the fourth oligonucleotide, the fifth oligonucleotide, and the sixth oligonucleotide results in a sixth overhang, wherein the first overhang, the second overhang, the third overhang, the fourth overhang, the fifth overhang, and the sixth overhang are in the target orientation,
and each of the plurality of dendrimers further comprises a sixth oligonucleotide dendron comprising an oligonucleotide stem linked to a plurality of oligonucleotide branches, with the oligonucleotide stem being hybridized to the sixth overhang, and
one or more oligonucleotides of the plurality of oligonucleotides of each of the sixth SNAs is hybridized to one or more oligonucleotide branches of the plurality of oligonucleotide branches of the sixth oligonucleotide dendron.

37. The crystal of claim 36, further comprising a plurality of seventh spherical nucleic acids (SNAs) comprising a nanoparticle core and a plurality of oligonucleotides attached to the external surface of the nanoparticle core, wherein:
the template core of each of the plurality of oligonucleotide dendrimers comprises a seventh oligonucleotide and hybridization of the first oligonucleotide, the second oligonucleotide, the third oligonucleotide, the fourth oligonucleotide, the fifth oligonucleotide, the sixth oligonucleotide, and the seventh oligonucleotide results in a seventh overhang, wherein the first overhang, the second overhang, the third overhang, the fourth overhang, the fifth overhang, the sixth overhang, and the seventh overhang are in the target orientation,
and each of the plurality of dendrimers further comprises a seventh oligonucleotide dendron comprising an oligonucleotide stem linked to a plurality of oligonucleotide branches, with the oligonucleotide stem being hybridized to the seventh overhang, and
one or more oligonucleotides of the plurality of oligonucleotides of each of the seventh SNAs is hybridized to one or more oligonucleotide branches of the plurality of oligonucleotide branches of the seventh oligonucleotide dendron.

38. The crystal of any one of claims 29-37, wherein the first oligonucleotide dendron, the second oligonucleotide dendron, the third oligonucleotide dendron, the fourth oligonucleotide dendron, the fifth oligonucleotide dendron, the sixth oligonucleotide dendron, and/or the seventh oligonucleotide dendron is a DNA dendron, a RNA dendron, a modified oligonucleotide dendron, or a combination thereof.

39. The oligonucleotide dendrimer of any one of claims 1-28, or the crystal of any one of claims 29-38, wherein the first oligonucleotide dendron, the second oligonucleotide dendron, the third oligonucleotide dendron, and/or the fourth oligonucleotide dendron each comprises about 2 to about 27 oligonucleotide branches.

40. The oligonucleotide dendrimer of any one of claim 1-28 or 39, or the crystal of any one of claims 29-39, wherein the first oligonucleotide dendron, the second oligonucleotide dendron, the third oligonucleotide dendron, and/or the fourth oligonucleotide dendron each comprises 6 branches.

41. The oligonucleotide dendrimer of any one of claim 1-28 or 39, or the crystal of any one of claims 29-39, wherein the first oligonucleotide dendron, the second oligonucleotide dendron, the third oligonucleotide dendron, and/or the fourth oligonucleotide dendron each comprises 9 branches.

42. The oligonucleotide dendrimer of any one of claim 1-28 or 39-41, or the crystal of any one of claims 29-41, wherein the first oligonucleotide dendron, the second oligonucleotide dendron, the third oligonucleotide dendron, and/or the fourth oligonucleotide dendron is a DNA dendron, a RNA dendron, a modified oligonucleotide dendron, or a combination thereof.

43. A method of forming a crystal, comprising:
contacting a plurality of oligonucleotide dendrimers with a plurality of first and second spherical nucleic acids (SNAs) such that the plurality of oligonucleotide dendrimers and the plurality of first and second SNAs hybridize to thereby arrange the first and second SNAS to form the crystal, wherein:
the plurality of oligonucleotide dendrimers each comprises:
a template core comprising first and second oligonucleotides hybridized to each other such that the template core has first and second overhangs, wherein the first and second overhangs are in a target orientation,
a first oligonucleotide dendron comprising an oligonucleotide stem linked to a plurality of oligonucleotide branches, with the oligonucleotide stem being hybridized to the first overhang, and
a second oligonucleotide dendron comprising an oligonucleotide stem linked to a plurality of oligonucleotide branches, with the oligonucleotide stem being hybridized to the second overhang,
each of the plurality of first SNAs comprises a nanoparticle core and a plurality of oligonucleotides attached to an external surface of the nanoparticle core,
each of the plurality of second SNAs comprises a nanoparticle core and a plurality of oligonucleotides attached to an external surface of the nanoparticle core,
upon contact:
one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers, and
one or more oligonucleotides of the plurality of oligonucleotides of each of the second SNAs is hybridizes to one or more oligonucleotide branches of the plurality of oligonucleotide branches of at least one second oligonucleotide dendron of the plurality of oligonucleotide dendrimers.

44. The method of claim 43, wherein in the target orientation the first overhang and the second overhang are oppositely disposed.

45. A method of forming a crystal, comprising:
contacting a plurality of oligonucleotide dendrimers with a plurality of first, second, and third spherical nucleic acids (SNAs) such that the plurality of oligonucleotide dendrimers and the plurality of first, second, and third SNAs hybridize to thereby arrange the first, second, and third SNAs to form the crystal, wherein:
the plurality of oligonucleotide dendrimers each comprises:
a template core comprising first, second, and third oligonucleotides hybridized to each other such that the template core has first, second, and third overhangs, wherein the first overhang, the second overhang, and the third overhang are in a target orientation,
a first oligonucleotide dendron comprising an oligonucleotide stem linked to a plurality of oligonucleotide branches, with the oligonucleotide stem being hybridized to the first overhang,
a second oligonucleotide dendron comprising an oligonucleotide stem linked to a plurality of oligonucleotide branches, with the oligonucleotide stem being hybridized to the second overhang, and
a third oligonucleotide dendron comprising an oligonucleotide stem linked to a plurality of oligonucleotide branches, with the oligonucleotide stem being hybridized to the third overhang,
each of the plurality of first SNAs comprises a nanoparticle core and a plurality of oligonucleotides attached to an external surface of the nanoparticle core,
each of the plurality of second SNAs comprises a nanoparticle core and a plurality of oligonucleotides attached to an external surface of the nanoparticle core,
each of the plurality of third SNAs comprises a nanoparticle core and a plurality of oligonucleotides attached to an external surface of the nanoparticle core,
upon contact:
one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers,
one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers, and
one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers.

46. The method of claim 45, wherein in the target orientation the first overhang, the second overhang, and the third overhang are in a radial orientation.

47. A method of forming a crystal, comprising:
contacting a plurality of oligonucleotide dendrimers with a plurality of first, second, third, and fourth spherical nucleic acids (SNAs) such that the plurality of oligonucleotide dendrimers and the plurality of first, second, third, and fourth SNAs hybridize to thereby arrange the first, second, third, and fourth SNAs to form the crystal, wherein:
the plurality of oligonucleotide dendrimers each comprises:
a template core comprising first, second, third, and fourth oligonucleotides hybridized to each other such that the template core has first, second, third, and fourth overhangs, wherein the first overhang, the second overhang, the third overhang, and the fourth overhang are in a target orientation,
a first oligonucleotide dendron comprising an oligonucleotide stem linked to a plurality of oligonucleotide branches, with the oligonucleotide stem being hybridized to the first overhang,
a second oligonucleotide dendron comprising an oligonucleotide stem linked to a plurality of oligonucleotide branches, with the oligonucleotide stem being hybridized to the second overhang, and
a third oligonucleotide dendron comprising an oligonucleotide stem linked to a plurality of oligonucleotide branches, with the oligonucleotide stem being hybridized to the third overhang,
a fourth oligonucleotide dendron comprising an oligonucleotide stem linked to a plurality of oligonucleotide branches, with the oligonucleotide stem being hybridized to the fourth overhang,
each of the plurality of first SNAs comprises a nanoparticle core and a plurality of oligonucleotides attached to an external surface of the nanoparticle core,
each of the plurality of second SNAs comprises a nanoparticle core and a plurality of oligonucleotides attached to an external surface of the nanoparticle core,
each of the plurality of third SNAs comprises a nanoparticle core and a plurality of oligonucleotides attached to an external surface of the nanoparticle core,
each of the plurality of fourth SNAs comprises a nanoparticle core and a plurality of oligonucleotides attached to an external surface of the nanoparticle core,
upon contact:
one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers,
one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers,
one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers, and
one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers.

48. The method of claim 47, wherein in the target orientation the first overhang and the second overhang are oppositely disposed, and the third overhang and the fourth overhang are oppositely disposed.

49. A method of forming a crystal, comprising:
contacting a plurality of oligonucleotide dendrimers with a plurality of first, second, third, fourth, and fifth spherical nucleic acids (SNAs) such that the plurality of oligonucleotide dendrimers and the plurality of first, second, third, fourth, and fifth SNAs hybridize to thereby arrange the first, second, third, fourth, and fifth SNAs to form the crystal, wherein:
the plurality of oligonucleotide dendrimers each comprises:
a template core comprising first, second, third, fourth, and fifth oligonucleotides hybridized to each other such that the template core has first, second, third, fourth, and fifth overhangs, wherein the first overhang, the second overhang, the third overhang, the fourth overhang, and the fifth overhang are in a target orientation,
a first oligonucleotide dendron comprising an oligonucleotide stem linked to a plurality of oligonucleotide branches, with the oligonucleotide stem being hybridized to the first overhang,
a second oligonucleotide dendron comprising an oligonucleotide stem linked to a plurality of oligonucleotide branches, with the oligonucleotide stem being hybridized to the second overhang, and
a third oligonucleotide dendron comprising an oligonucleotide stem linked to a plurality of oligonucleotide branches, with the oligonucleotide stem being hybridized to the third overhang,
a fourth oligonucleotide dendron comprising an oligonucleotide stem linked to a plurality of oligonucleotide branches, with the oligonucleotide stem being hybridized to the fourth overhang,
a fifth oligonucleotide dendron comprising an oligonucleotide stem linked to a plurality of oligonucleotide branches, with the oligonucleotide stem being hybridized to the fifth overhang,
each of the plurality of first SNAs comprises a nanoparticle core and a plurality of oligonucleotides attached to an external surface of the nanoparticle core,
each of the plurality of second SNAs comprises a nanoparticle core and a plurality of oligonucleotides attached to an external surface of the nanoparticle core,
each of the plurality of third SNAs comprises a nanoparticle core and a plurality of oligonucleotides attached to an external surface of the nanoparticle core,
each of the plurality of fourth SNAs comprises a nanoparticle core and a plurality of oligonucleotides attached to an external surface of the nanoparticle core,
each of the plurality of fifth SNAs comprises a nanoparticle core and a plurality of oligonucleotides attached to an external surface of the nanoparticle core,
upon contact:
one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers,
one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers,
one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers,
one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers, and
one or more oligonucleotides of the plurality of oligonucleotides of each of the fifth SNAs hybridizes to one or more oligonucleotide branches of the plurality of oligonucleotide branches of at least one fifth oligonucleotide dendron of the plurality of oligonucleotide dendrimers.

50. A method of forming a crystal, comprising:
contacting a plurality of oligonucleotide dendrimers with a plurality of first, second, third, fourth, fifth, and sixth spherical nucleic acids (SNAs) such that the plurality of oligonucleotide dendrimers and the plurality of first, second, third, fourth, fifth, and sixth SNAs hybridize to thereby arrange the first, second, third, fourth, fifth, and sixth SNAs to form the crystal, wherein:
the plurality of oligonucleotide dendrimers each comprises:
a template core comprising first, second, third, fourth, fifth, and sixth oligonucleotides hybridized to each other such that the template core has first, second, third, fourth, fifth, and sixth overhangs, wherein the first overhang, the second overhang, the third overhang, the fourth overhang, the fifth overhang, and the sixth overhang are in a target orientation,
a first oligonucleotide dendron comprising an oligonucleotide stem linked to a plurality of oligonucleotide branches, with the oligonucleotide stem being hybridized to the first overhang,
a second oligonucleotide dendron comprising an oligonucleotide stem linked to a plurality of oligonucleotide branches, with the oligonucleotide stem being hybridized to the second overhang, and
a third oligonucleotide dendron comprising an oligonucleotide stem linked to a plurality of oligonucleotide branches, with the oligonucleotide stem being hybridized to the third overhang,
a fourth oligonucleotide dendron comprising an oligonucleotide stem linked to a plurality of oligonucleotide branches, with the oligonucleotide stem being hybridized to the fourth overhang,
a fifth oligonucleotide dendron comprising an oligonucleotide stem linked to a plurality of oligonucleotide branches, with the oligonucleotide stem being hybridized to the fifth overhang,
a sixth oligonucleotide dendron comprising an oligonucleotide stem linked to a plurality of oligonucleotide branches, with the oligonucleotide stem being hybridized to the sixth overhang,
each of the plurality of first SNAs comprises a nanoparticle core and a plurality of oligonucleotides attached to an external surface of the nanoparticle core,
each of the plurality of second SNAs comprises a nanoparticle core and a plurality of oligonucleotides attached to an external surface of the nanoparticle core,
each of the plurality of third SNAs comprises a nanoparticle core and a plurality of oligonucleotides attached to an external surface of the nanoparticle core,
each of the plurality of fourth SNAs comprises a nanoparticle core and a plurality of oligonucleotides attached to an external surface of the nanoparticle core,
each of the plurality of fifth SNAs comprises a nanoparticle core and a plurality of oligonucleotides attached to an external surface of the nanoparticle core,
each of the plurality of sixth SNAs comprises a nanoparticle core and a plurality of oligonucleotides attached to an external surface of the nanoparticle core,
upon contact:
one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers,
one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers,
one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers,
one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers,
one or more oligonucleotides of the plurality of oligonucleotides of each of the fifth SNAs hybridizes to one or more oligonucleotide branches of the plurality of oligonucleotide branches of at least one fifth oligonucleotide dendron of the plurality of oligonucleotide dendrimers, and
one or more oligonucleotides of the plurality of oligonucleotides of each of the sixth SNAs hybridizes to one or more oligonucleotide branches of the plurality of oligonucleotide branches of at least one sixth oligonucleotide dendron of the plurality of oligonucleotide dendrimers.

51. A method of forming a crystal, comprising:
contacting a plurality of oligonucleotide dendrimers with a plurality of first, second, third, fourth, fifth, sixth, and seventh spherical nucleic acids (SNAs) such that the plurality of oligonucleotide dendrimers and the plurality of first, second, third, fourth, fifth, sixth, and seventh SNAs hybridize to thereby arrange the first, second, third, fourth, fifth, sixth, and seventh SNAs to form the crystal, wherein:
the plurality of oligonucleotide dendrimers each comprises:
a template core comprising first, second, third, fourth, fifth, sixth, and seventh oligonucleotides hybridized to each other such that the template core has first, second, third, fourth, fifth, sixth, and seventh overhangs, wherein the first overhang, the second overhang, the third overhang, the fourth overhang, the fifth overhang, the sixth overhang, and the seventh are in a target orientation,
a first oligonucleotide dendron comprising an oligonucleotide stem linked to a plurality of oligonucleotide branches, with the oligonucleotide stem being hybridized to the first overhang,
a second oligonucleotide dendron comprising an oligonucleotide stem linked to a plurality of oligonucleotide branches, with the oligonucleotide stem being hybridized to the second overhang, and
a third oligonucleotide dendron comprising an oligonucleotide stem linked to a plurality of oligonucleotide branches, with the oligonucleotide stem being hybridized to the third overhang,
a fourth oligonucleotide dendron comprising an oligonucleotide stem linked to a plurality of oligonucleotide branches, with the oligonucleotide stem being hybridized to the fourth overhang,
a fifth oligonucleotide dendron comprising an oligonucleotide stem linked to a plurality of oligonucleotide branches, with the oligonucleotide stem being hybridized to the fifth overhang,
a sixth oligonucleotide dendron comprising an oligonucleotide stem linked to a plurality of oligonucleotide branches, with the oligonucleotide stem being hybridized to the sixth overhang,
a seventh oligonucleotide dendron comprising an oligonucleotide stem linked to a plurality of oligonucleotide branches, with the oligonucleotide stem being hybridized to the seventh overhang,
each of the plurality of first SNAs comprises a nanoparticle core and a plurality of oligonucleotides attached to an external surface of the nanoparticle core,
each of the plurality of second SNAs comprises a nanoparticle core and a plurality of oligonucleotides attached to an external surface of the nanoparticle core,
each of the plurality of third SNAs comprises a nanoparticle core and a plurality of oligonucleotides attached to an external surface of the nanoparticle core,
each of the plurality of fourth SNAs comprises a nanoparticle core and a plurality of oligonucleotides attached to an external surface of the nanoparticle core,
each of the plurality of fifth SNAs comprises a nanoparticle core and a plurality of oligonucleotides attached to an external surface of the nanoparticle core,
each of the plurality of sixth SNAs comprises a nanoparticle core and a plurality of oligonucleotides attached to an external surface of the nanoparticle core,
each of the plurality of seventh SNAs comprises a nanoparticle core and a plurality of oligonucleotides attached to an external surface of the nanoparticle core,
upon contact:
one or more oligonucleotides of the plurality of oligonucleotides of each of the first SNAs hybridizes to one or more oligonucleotide branches of the plurality of oligonucleotide branches of at least one first oligonucleotide dendron of the plurality of oligonucleotide dendrimers,
one or more oligonucleotides of the plurality of oligonucleotides of each of the second SNAs hybridizes to one or more oligonucleotide branches of the plurality of oligonucleotide branches of at least one second oligonucleotide dendron of the plurality of oligonucleotide dendrimers,
one or more oligonucleotides of the plurality of oligonucleotides of each of the third SNAs hybridizes to one or more oligonucleotide branches of the plurality of oligonucleotide branches of at least one third oligonucleotide dendron of the plurality of oligonucleotide dendrimers,
one or more oligonucleotides of the plurality of oligonucleotides of each of the fourth SNAs hybridizes to one or more oligonucleotide branches of the plurality of oligonucleotide branches of at least one fourth oligonucleotide dendron of the plurality of oligonucleotide dendrimers,
one or more oligonucleotides of the plurality of oligonucleotides of each of the fifth SNAs hybridizes to one or more oligonucleotide branches of the plurality of oligonucleotide branches of at least one fifth oligonucleotide dendron of the plurality of oligonucleotide dendrimers, and
one or more oligonucleotides of the plurality of oligonucleotides of each of the sixth SNAs hybridizes to one or more oligonucleotide branches of the plurality of oligonucleotide branches of at least one sixth oligonucleotide dendron of the plurality of oligonucleotide dendrimers, and
one or more oligonucleotides of the plurality of oligonucleotides of each of the seventh SNAs hybridizes to one or more oligonucleotide branches of the plurality of oligonucleotide branches of at least one seventh oligonucleotide dendron of the plurality of oligonucleotide dendrimers.

52. The method of claim 43 or claim 44, wherein each of the plurality of oligonucleotide dendrimers is the oligonucleotide dendrimer of any one of claim 1-28 or 39-42.

53. The method of claim 45 or claim 46, wherein each of the plurality of oligonucleotide dendrimers is the oligonucleotide dendrimer of any one of claim 5-28 or 39-42.

54. The method of claim 47 or claim 48, wherein each of the plurality of oligonucleotide dendrimers is the oligonucleotide dendrimer of any one of claim 10-28 or 39-42.

55. The method of claim 49, wherein each of the plurality of oligonucleotide dendrimers is the oligonucleotide dendrimer of any one of claim 26-28 or 39-42.

56. The method of claim 50, wherein each of the plurality of oligonucleotide dendrimers is the oligonucleotide dendrimer of any one of claim 27-28 or 39-42.

57. The method of claim 51, wherein each of the plurality of oligonucleotide dendrimers is the oligonucleotide dendrimer of claim 28 or 39-42.

58. The method of claim 43 or claim 44, wherein the crystal is the crystal of any one of claims 29-42.

59. The method of claim 45 or claim 46, wherein the crystal is the crystal of any one of claims 31-42.

60. The method of claim 47 or claim 48, wherein the crystal is the crystal of any one of claims 33-42.

61. The method of claim 49, wherein the crystal is the crystal of any one of claims 35-42.

62. The method of claim 50, wherein the crystal is the crystal of any one of claims 36-42.

63. The method of claim 51, wherein the crystal is the crystal of any one of claims 37-42.

64. The method of any one of claims 43-63, further comprising contacting the crystal with a chemical input, thereby resulting in at least partial disassembly of the crystal.

65. The method of claim 64, wherein the chemical input is toe-hold mediated strand displacement, a light responsive chemistry, a pH responsive chemistry, or a combination thereof.
